(19) United States
(12) Patent Application Publication
(10) Pub. No.: US 2010/0093659 A1
(43) Pub. Date: Apr. 15, 2010

(54) POLYVALENT BIOCONJUGATES

(75) Inventors: Jari Natunen, Vantaa (FI); Jari Helin, Vantaa (FI); Krista Weikkolainen, Helsinki (FI)

Correspondence Address: BROOKS KUSHMAN P.C., 1000 TOWN CENTER, TWENTY-SECOND FLOOR, SOUTHFIELD, MI, 48075, US

(73) Assignee: GLYKOS FINLAND OY, Helsinki (FI)

(21) Appl. No.: 12/519,304

(22) PCT Filed: Dec. 13, 2007
(86) PCT No.: PCT/FI07/50687

§ 371 Date: Jun. 15, 2009

(30) Foreign Application Priority Data
Dec. 13, 2006 (FI) 20065800

(51) Int. Cl. A61K 31/715; C08B 37/00; C08B 37/10; C08B 37/16; C08B 37/08; C07H 1/00; A61P 31/00

(52) U.S. Cl. 514/54; 536/123.1; 536/55.1; 536/21; 536/46; 536/20; 536/124

(57) ABSTRACT

The present invention is directed to conjugates for biorecognition comprising (i) an carbohydrate backbone structure (PO) of 5 to 20 monosaccharide units, (ii) oligosaccharide biorecognition groups (Bio) of 1 to 10 monomer units, (iii) a bifunctional spacer groups of the formula -(y)p-(S)q-(z)r-, wherein S is a spacer group, p, q and r are each 0 or 1, whereby at least one of p and r is different from 0, and y and z are chemoselective ligation groups, which covalently link a said Bio group to said backbone structure, and the degree of conjugation, indicating the average number of covalently attached Bio biorecognition groups per monomer unit of the backbone, being from 0.2 to 1. The invention is also directed to processes for their preparation, intermediates for use in the process as well as use of said conjugates, especially for inhibiting pathogenic bacteria.

### FIELD OF THE INVENTION

The present invention relates to conjugates for biorecognition purposes, especially for use in medicine. Especially the invention relates to carbohydrate polymer structures comprising biorecognition groups which are coupled to an oligosaccharide carrier or backbone by chemoselective ligation. In the present invention, special linking chemistries are used to allow the linking of a biorecognition group effectively and specifically to the backbone. In addition, the invention is directed to the use of said conjugates.

### BACKGROUND OF THE INVENTION

Some 6-position amine derivatives of cyclodextrins are known, and the present invention is especially directed to different cyclodextrin conjugates, with effective and useful carbonyl chemistry (one synthesis step less etc.), the invention further revealed conjugates of chitosan, wherein carbohydrates are not linked from the reducing end as disclosed in some prior art publications, such as WO99/45032 and WO2004/085487.

Carbohydrates are widely expressed on cell surfaces where they form an important class of biological recognition molecules. The multilateral importance of glycosylated structures ranges from beneficial biological events, such as tissue development, cell division processes, and immune response to detrimental disease processes, such as pathogen homing on their target tissues, cancer metastasis, and inflammation (Davis, 2000; Dwek, 1996; Gabius, 1997; L is & Sharon, 1998; Varki, 1993). The role of carbohydrates as cell surface receptors enabling adherence of bacteria, viruses, and parasites in the early stages of infection has in recent years gained growing therapeutic interest. Inhibition of pathogen-host recognition/interaction using carbohydrate-based pharmaceuticals is under intensive development and presents a promising approach for the prevention of susceptible microbial infections.

The relatively weak affinity between carbohydrates and their receptors is often overcome by adopting multivalent binding, in which carbohydrate recognition domains in receptors (lectins) are clustered, as was recently illustrated and summarized for mammalian lectins (Gabius, Andre, Kaltner & Siebert, 2002). This generates challenges for successful development of carbohydrate based pharmaceuticals. To mimic the natural multivalent presentation, a number of scaffolds have been used. Among the carriers employed for constructing multivalent conjugates, the most common are dendrimers (Rockendorf & Lindhorst, 2001), cyclodextrins (Roy, Hernandez-Mateo & Santoyo-Gonzalez, 2000), calixarenes (Dondoni, Marra, Scherrmann, Casnati, Sansone & Ungaro, 1997), and neoglycoconjugates (Roy, 1996). As an example, it has been shown that single intranasal inoculations with polyacrylamide-based conjugates bearing sialylated N-glycans increase the survival of mice experimentally infected with influenza viruses, probably by binding to the virus hemagglutinin and thus decreasing the virus infectivity (Gambaryan, Tuzikov, Chinarev, Juneja, Bovin & Matrosovich, 2002). In addition, adhesion of Helicobacter pylori was inhibited to gastrointestinal epithelial cells by monomeric 3′-sialyllactose at millimolar concentrations whereas multivalent neoglycoproteins bearing 3′-sialyllactose were 1000-fold more potent (Simon, Goode, Mobasseri & Zopf, 1997).

Details of multivalent protein-carbohydrate interactions are not well understood making an empirical approach to the development of multivalent ligands necessary. There is therefore a growing need for development of new linking chemistry on scaffolds already in use as well as for new types of structural scaffolds. As part of our ongoing project on the development of multivalent carbohydrate analogs we have focused on synthesis of multivalent glycoconjugates based on carbohydrate scaffolds. Carbohydrate scaffolds in general may offer better biocompatibility, as they exhibit excellent solubility in water and low antigenicity. Carbohydrate based multivalent conjugates previously described include cyclodextrins (Fulton & Stoddart, 2001; Houseman & Mrksich, 2002; Matsuda et al., 1997; Ortiz Mellet, Defaye & Fernandez, 2002), hyaluronic acid (Soltés et al., 1999), chitosan (Sakagami, Horie, Nakamoto, Kawaguchi & Hamana, 2000), and heparin (Sakagami et al., 2000).

In addition, multivalent neoglycoproteins, polymers, liposomes, and dendrimers, functionalized with sialic acid capable of blocking influenza virus—cell attachment, have been described (Roy, R. (1996) Syntheses and some applications of chemically defined multivalent glycoconjugates. Curr Opin Struct Biol 6, 692-702.). Another exciting area for glycoconjugates is their use as postal codes in vectorized drug delivery.

Polyvalent conjugates comprising a carrier structure in the form of, for example, a carbohydrate or a polypeptide carrying covalently attached various kinds of structures, including biorecognition structures, for example oligosaccharides, are known. Thus for example the U.S. Pat. No. 6,037,467 discloses structures comprising hydrophilic carbohydrates covalently attached over a bifunctional spacer to a hydrophilic polymer, for example chitosan, heparin, hyaluronic acid, or starch, including in addition a potentiator to potentiate the effect.

### SUMMARY OF THE INVENTION

The present invention is directed to novel polyvalent conjugate structures comprising

(i) a carbohydrate backbone structure (PO) of 5 to 20 monomer units,
(ii) oligosaccharide biorecognition groups (Bio) of 1 to 10 monomer units,
(iii) a bifunctional spacer groups of the formula -(y)ₚ-(S)_{q}-(z)ᵣ-, wherein S is a spacer group, p, q and r are each 0 or 1, whereby at least one of p and r is different from 0, and y and z are chemoselective ligation groups, of which y is covalently linked to a said Bio group and z is covalently linked to the said backbone structure and the degree of conjugation, defining the average number of covalently attached Bio biorecognition groups per monomer unit of the backbone, being from 0.2 to 1.

The degree of conjugation indicates on an average the number of biorecognition groups per monomer unit of the backbone, whereby a degree of conjugation of for example 0.2 means on an average 2 biorecognition groups per 10 monomer units, and a degree of conjugation of 1 means on an average one biorecognition group in each monomer unit of the backbone. Preferably the degree of conjugation is about 0.2-1, more preferably 0.3-0.7, and most preferably 0.4-0.6.

The present invention is thus directed to conjugates comprising a carrier or backbone structure and attached biorecognition groups, for biorecognition purposes, which conjugates carry, covalently attached to the backbone PO, groups of the formula Bio-(y)ₚ-(S)_{q}-(z)ᵣ- wherein the symbols have the meanings indicated above.

The polyvalent bioconjugates as defined above can also be expressed with the formula

[Bio-(y)ₚ-(S)_{q}-(z)ᵣ-]ₙ[Z]ₘPO (I)

wherein PO, Bio, y, S, z, p, q and r have the meaning given above, n indicates the number of biorecognition groups in the conjugate, Z can have the meaning of (y′)ₚ-(S)_{q}-(z)ᵣ- or of a group z′, wherein y′ means a group that can form the linkage y, and z′ means a group on PO that can form the linkage z, and m is an integer which is >0 so that (n+m) is equal to or less. Preferably m is 0, meaning that the conjugate product does not essentially contain any species with incompletely reacted spacer groups or fractions thereof.

According to an embodiment, the conjugates have the formula

{Hex2(X)ₚ₁}[aHex(X)ₚ₂b{Hex2(X)ₚ₃}ₚ₄]ₙ₁aHex(X)ₚ₅—R (Ia)

wherein Hex and Hex2 are each a hexose group which comprises a group for bonding to X, X is a bioactive conjugate according to the formula:

Bio-(y)ₚ-(S)_{q}-(z)ᵣ-

wherein Bio, S, y, z, p, q, and r have the meaning given in the claim I, n1 is an integer >1, each of p1, p2, p3, p4 and p5 are 0 or 1, provided that at least one ??? of p1, p2, p3, p4 and p5 is different from 0,
a and b are the anomeric linkages of the monosaccharide Hex2 and Hex respectively, the linkage positions being either α or β1-4/1-3
R is a derivatization group at the reducing end of the saccharide, or a modified reducing end, such as reduced monosaccharide residue, an alditol, or anhydromannitol.

The present invention is also directed to processes for the preparation of the conjugates according to the invention, as well as to their use, especially for the inhibition of the binding of pathogenic bacteria.

### BRIEF DESCRIPTION OF THE DRAWING

FIGS. 1A and 1B. (A) MALDI TOF mass spectrum of a chondroitin 14-mer (Compound 2) isolated by gel filtration chromatography from CSA hydrolysate. See text for peak assignements. (B) MALDI-TOF mass spectrum of LNnT glycosylamine derivatised chondroitin 14-mer (Compound 3). Representative signals are indicated and the proposed structures are given in the inset.

FIGS. 2A, 2B and 2C. 1D-¹H-NMR spectra of (A) LNnT-NH-Ch14 (Compound 3). (B) LNnT-NH-ox-γ-CD (Compound 6). (C) LNnT-Aoa-γ-CD (Compound 9). See Scheme 1 and 2 in FIGS. 5 and 6 for more structural details.

FIGS. 3A and 3B. (A) MALDI-TOF mass spectrum of LNnT glycosylamine derivatized ox-γ-CD (Compound 6). (B) MALDI-TOF mass spectrum of γ-CD derivatized with LNnT through an oxime-linkage (Compound 9). Representative signals are indicated and the proposed structures are given in the inset.

FIG. 4. Analysis of oxime-bond stability under acidic conditions. LNnT-Aoa was incubated under acidic conditions at room temperature and at +37° C. The relative amounts of LNnT-Aoa and the breakdown product LNnT were analyzed at different time points by MALDI-TOF MS.

FIG. 5. Scheme 1. (a) desulphation: 90% DMSO-10% MeOH, 80° C., 5 h; (b) hydrolysis: 0.5 M TFA, 60° C., 20 h; (c) amidation: LNnT-NH₂, HBTU, DIPEA, pyridine, room temperature, 4 days.

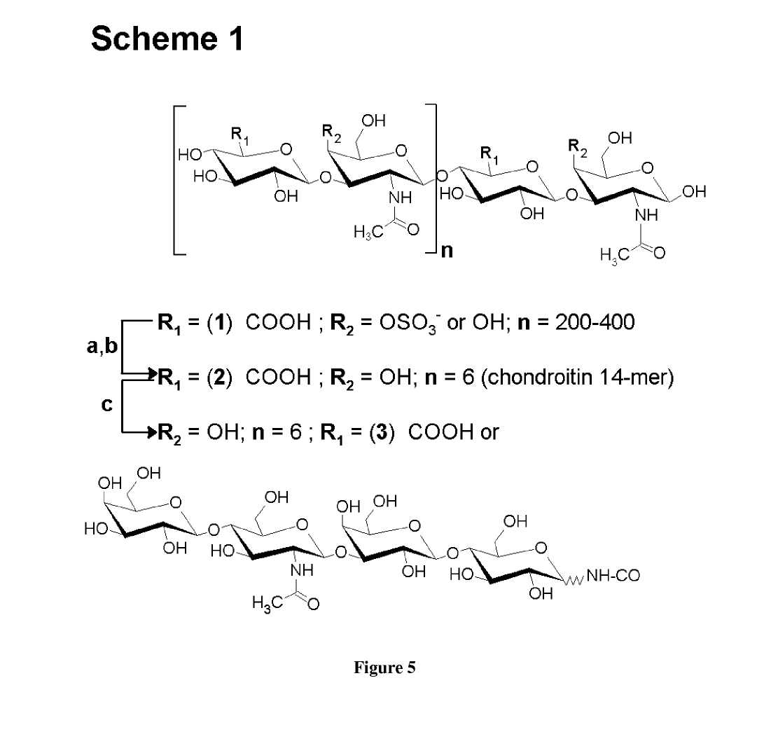

FIG. 6. Scheme 2. (a) oxidation: TEMPO, NaBr, NaClO, 0.2 M Na-carbonate buffer, pH 10, on ice, (remaining aldehyde groups reduced by NaBH₄, on ice, 1 h); (b) amidation: LNnT-NH₂, HBTU, DIPEA, pyridine, room temperature, 4 days; (c) esterification: Boc-Amoc-HAc, HBTU, DIPEA, pyridine, room temperature, 2 days; (d) Boc removal: TFA, room temperature, 10 min; (e) oxime ligation: LNnT, 0.2 M Na-acetate pH 4, room temperature, 15 h.

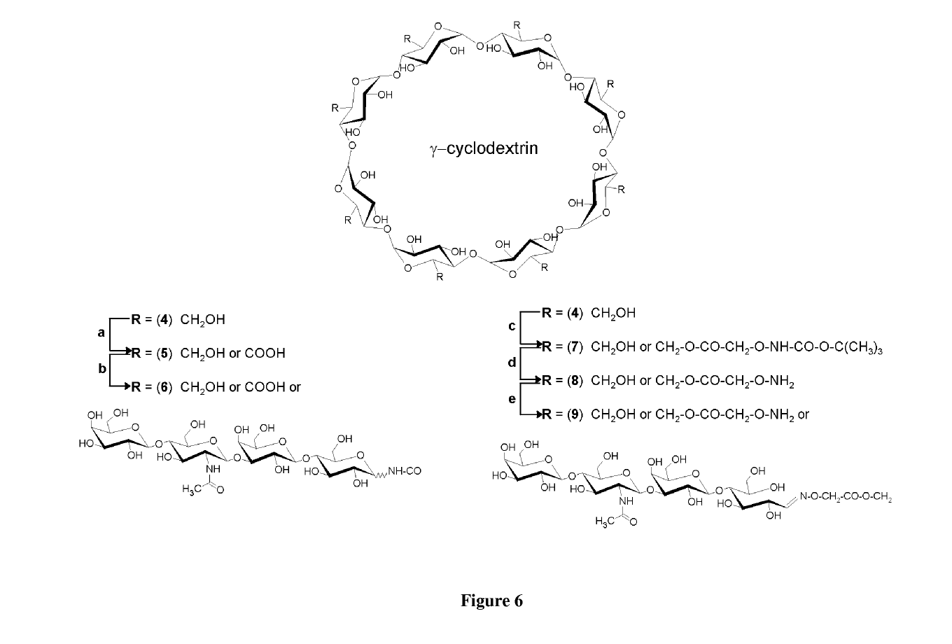

FIG. 7. Modification of LNnT using aminooxyacetic acid and amidation to DAP-ox-γ-CD.

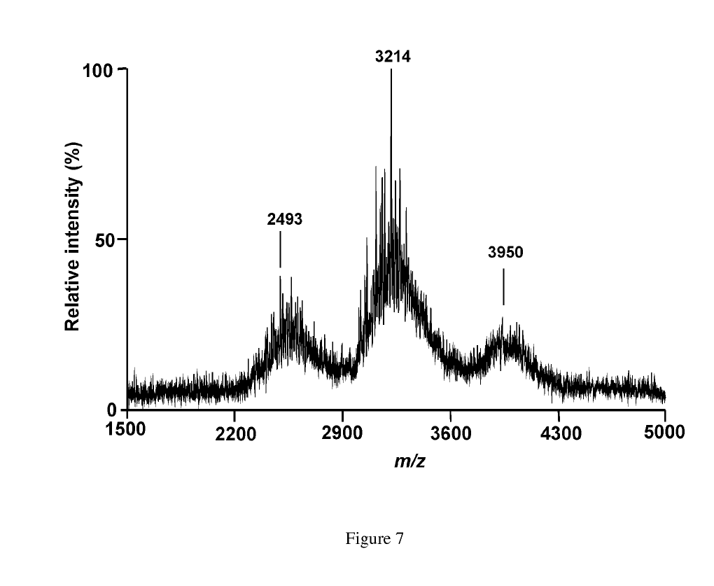

FIGS. 8A and 8B. (A) MALDI-TOF mass spectrum of chondroitin 14-mer fraction prepared by acid hydrolysis. The signals were identified as chondroitin 12-mer (m/z 2293.1 [M-H]⁻, 2373.1 [M-H+SO₃]⁻), chondroitin 14-mer (m/z 2672.7 [M-H]⁻, 2752.9 [M-H+SO₃]⁻, 2630.4 [M-H−Ac]⁻, 2710.0 [M-H+SO₃−Ac]⁻), and chondroitin 16-mer (m/z 3052.0 [M-H]⁻, 3010.0 [M-H−Ac]⁻. In addition, minor signals representing chondroitin 13-mer (GalNAc₇GlcA₆) m/z 2496.5 [M-H]⁻, and 2576.5 [M-H+SO₃]⁻ were observed. (B) MALDI-TOF mass spectrum of LNDFH I-DAP-Ch14 conjugate, obtained by reductive amination of LNDFH I (Lewis-b hexasaccharide) and diaminopropane modified chondroitin 14-mer fraction. Representative signals are indicated and the proposed structures are given in the inset (LNDFH I marked as Leb). The heterogeneity in the conjugate signals is due to chondroitin backbones of different sizes as well as variable level of amidation.

FIGS. 9A and 9B. Anomeric regions of 1D-¹H-NMR spectra of (A) LNDFH I-DAP-Ch14 (Compound 4a of FIG. 11) with pNP-β-GlcA as internal quantification standard. (B) LNnT-DAP-ox-γ-CD (Compound 8 of FIG. 12). See Scheme 3 and 4 of FIGS. 11 and 12 for more structural details.

FIGS. 10A and 10B. (A) MALDI-TOF mass spectrum of LNnT-DAP-ox-γ-CD conjugate. (B) MALDI-TOF mass spectrum of sialylated LNnT-DAP-ox-γ-CD conjugate. Representative signals are indicated and the proposed structures are given in the inset. The heterogeneity in the conjugate signals is due to variations in γ-CD scaffold oxidation, amidation and N-acetylation.

FIG. 11. Scheme 3. (a) desulphation, (b) hydrolysis, (c) 1,3-diaminopropane amidation, (d) reductive amination.

FIG. 12. Scheme 4. (a) oxidation, (b) 1,3-diaminopropane amidation, (c) reductive amination.

### DETAILED DESCRIPTION OF THE INVENTION

The present invention seeks to solve the problem of conjugation of a biorecognition molecule to an oligomeric carrier when both the carrier and the biorecognition molecule are non-protected molecules with multiple functional groups. Thus the invention can take advantage of the fact that numerous natural biorecognition molecules can be produced biosynthetically in non-protected forms.

The biorecognition molecules for use in the invention are preferably recognized by a receptor in a medically, therapeutically or nutritionally important context. In a preferred embodiment, the polyvalent constructs are therapeutical molecules for prophylaxis of a disease or for the treatment of an active disease. The constructs can thus be used in medicines or in functional foods or as food additives or nutritional supplements to prevent diseases in vivo. Moreover, the present invention is directed to the use of polyvalent presentation molecules in various therapeutical or consumer products or for neutralization of pathogenic agents such as bacteria, toxins, lectins, or enzymes such as glycosidases, proteases or harmful antibodies. The present invention is also directed to the use of the polyvalent constructs in analytics, as well as for the purification of receptors binding to the polyvalent constructs.

The present invention is specifically directed to safe polyvalent constructs for in vivo uses. The polyvalent constructs are designed to be non-immunogenic or essentially non-immunogenic.

### Preferred Backbone Structures

According to the invention the preferred backbone structure comprises known and acceptable biocompatible molecules. The backbone structure comprises large oligosaccharide structures comprising from 5-20 monosaccharide units, and it can be linear or it can be cyclic.

Preferred backbone structures include the following structures or suitably sized fragments or modified derivatives thereof: glycosaminoglycans, such as chondroitin, chondroitin sulphate, dermatan sulphate, poly-N-acetylactosamine or keratan sulphate, hyaluronic acid, heparin, and heparin precursors, including N-acetylheparosan and heparan sulphate; chitin, chitosan, starch and starch or glycogen fractions. Useful starch fractions includes amylose and amylopectin fractions.

It is realized that other biological carbohydrate polymers that carry hexuronic acid residues can be derivatized by the same chemistry described for chondroitin and oxidized gamma-cyclodextrin above. These include but are not restricted to large MW and fragments of hyaluronic acid, dermatan sulphate, alginic acid and pectin as well as oxidized derivatives of neutral, acidic and basic polysaccharides, e.g. starch, fucoidan and chitosan.

To carry out amidation of 1,3-diaminopropane to a polysaccharide with a carboxylic acid group, the polysaccharide is dissolved in a suitable solvent, e.g. 90% aqueous pyridin. To this solution is added a suitable molar excess (e.g. 100-fold) of 1,3-diaminopropane per carboxylic acid unit, carboxylic acid activator and a tertiary amine. Suitable carboxylic acid activators include e.g. HBTU, ByBUP and DMT-MM. Suitable tertiary amines include e.g. diisopropylethylamine and trimethylamine. The reaction is carried out for a suitable period of time and the solvent is removed e.g. by evaporation. The mixture is dissolved in aqueous solution that may contain e.g. methanol or ethanol to solubilize the less water soluble reactants. The small MW reactants are removed by e.g. dialysis or filtration through a filter of low MW cut-off.

The 1,3-diaminopropane derivatized polysaccharide can be derivatized by a reducing carbohydrate by reductive amination: The polysaccharide and the reducing carbohydrate are dissolved in an aqueous buffer, e.g. 0.1 M Na-borate pH 8.5, and a reductant is added. Suitable reductants are e.g. NaCNBH₄ and Na(Ac)₃BH. The small MW reactants are removed by e.g. dialysis or filtration through a filter of low MW cut-off.

Carbohydrates carrying primary amino groups can be directly coupled by amidation to polysaccharides with a carboxylic group. Reducing carbohydrates can be converted to glycosylamines carrying a primary amino group at the reducing terminus by incubation in ammonium bicarbonate. The glycosylamine form of carbohydrate is linked to any polysaccharide with an carboxylic group in a reaction similar to that described above for 1,3-diaminopropane. The modified polysaccharide can be purified by e.g. dialysis or filtration through a filter of low MW cut-off.

It is also realized that Boc-aminooxyacetic acid can be esterified to many polysaccharides. To carry out these reactions, the polysaccharide or a fragment of the polysaccharide is dissolved in a suitable dry solvent (e.g. pyridin or dimethylacetamide). To this solution Boc-aminooxyacetic acid, carboxylic acid activator and a tertiary amine are added. Suitable carboxylic acid activators include e.g. HBTU, ByBUP, and carbodiimide-type activators. Suitable tertiary amines include e.g. diisopropylethylamine and trimethylamine. The reaction is carried out for a suitable period of time and the solvent is removed e.g. by evaporation. The reaction mixture is dissolved in aqueous solution that may contain e.g. methanol or ethanol to solubilize the less water soluble reactants. The small MW reactants are removed by e.g. dialysis or filtration through a filter of low MW cut-off. The purified Boc-aminooxyacetic acid esterified polysaccharide or polysaccharide fragment can be treated with dry acid (e.g. TFA) to detach the protecting Boc-group. The aminooxyacetic acid side chains can be used to bind reducing oligosaccharides via an oxime-linkage to the derivatized polysaccharide molecule by incubating in e.g. sodium acetate buffer, pH 4.

For oligovalent presentation cyclic oligosaccharides called cyclodextrins are preferred, which have been accepted for in vivo applications. Such cyclodextrins include α-, β- and γ-cyclodextrins. A preferred cyclodextrin for use as a backbone is γ-cyclodextrin, which is a cyclic oligosaccharide sonsisting of 8 glycopyranose units joined together by α(1-4) linkages.

Other possible backbone carbohydrates to be used include cellulose oligosaccharides, pectin oligosaccharides, fucose polysaccharides, galactose comprising polysaccharides, xylose comprising polysaccharides, GalNAc or galactosamine-comprising polysaccharides and sialic acid polysaccharides. In its broadest embodiment, when natural glycosidic linkages are used between natural human type monosaccharide residues, natural or synthetic backbone carbohydrates are useful for use according to the invention. The possible degradation of the backbone carbohydrate by glycosidase enzymes is controlled or prevented by the number of biorecognition groups, such as oligosaccharides forming branches on the backbone.

In a preferred embodiment of the invention the backbone saccharide may be a homosaccharide consisting of a single major monosaccharide type or derivatives thereof. Alternatively the backbone saccharide is a heterosaccharide consisting of two types of monosaccharide residues. Examples of the monopolysaccharides include starch and pectin.

In a preferred embodiment of the invention the backbone saccharide may be a homosaccharide consisting of a single major monosaccharide type or derivatives thereof. Alternatively the backbone saccharide is a heterosaccharide consisting of two types of monosaccharide residues. Examples of the monopolysaccharides include

{Hex2(X)ₚ₁}[aHex(X)ₚ₂b{Hex2(X)ₚ₃}ₚ₄]ₙ₁aHex(X)ₚ₅- (Ia)

wherein Hex and Hex2 are each a hexose group which may typically comprise a carboxylic acid or amine group for bonding to X, X is a bioactive conjugate according to the formula:

Bio-(y)ₚ-(S)_{q}-(z)ᵣ-

wherein Bio, S, y, z, p, q, and r have the meaning given in the formula (I), n1 is an integer >1, each of p1, p2, p3, p4 and p5 are 0 or 1, provided that at least one of p1, p2, p3, p4 and p5 is different from 0,
a and b are the anomeric linkages of the monosaccharide Hex2 and Hex respectively, the linkage positions being either α or β1-4/1-3
R is a derivatization group at the reducing end of the polysaccharide, or a modified reducing end, such as reduced monosaccharide residue, an alditol, or anhydromannitol such as formed by degradation of chitosan by sodium nitrite and reduction by sodium borohydride.

In the above formula Hex and Hex2 are preferably independently of each other chosen from the group of GlcN, GlcNAc, GlcA, Glc, GalNAc, GalN, Gal, IdoA, GalA, Xyl, Man, sialic acid, Fuc, preferably GlcN, GlcNAc, GlcA, Glc, GalNAc, GalN, Gal, IdoA.

The present invention is specifically directed to conjugate structures according to the Formula

[(y′)ₚ-(S)_{q}-(z)ᵣ]ₙ-PO (II)

wherein n is an integer >1, S is a spacer group, y′ is an aminooxy group NH₂—O— or a chemoselective linking group and z is a O-hydroxylamine residue —O—NH— or —O—N═, with the nitrogen atom being linked to the PO structure, or a chemoselective linking group, there being at least one of aminooxy and O-hydroxylamine group present p, q and r are each 0 or 1, whereby at least one of p and r is different from 0, and PO is a linear polysaccharide or oligosaccharide or mixture thereof carrying n [(y′)ₚ-(S)_{q}-(z)ᵣ]ₙ- groups on the polymer backbone. Preferably n is at least two, more preferably at least three. In a preferred embodiment the said structures aim for oligovalent presentation and n is between 2-10. More preferably n is between 3 and 9.

Preferably S is an organic spacer residue with enough flexibility for effective coupling of the Bio-structures to be conjugated. In a preferred embodiment the organic spacer residue is an alkylene, or a polyether such as polyethylene glycol.

Specific preferred structures have the formulas:

a) [NHR″O—(S)_{q}—CO—O/NH]ₙ—PO (IIa)

or specifically

b) [NHR″O—(CH₂)_{q2}—CO—O/NH]ₙ—PO

wherein the symbols PO, S, q and n have the meaning given above in formula (II), q2 is an integer from 1 to 26, preferably from 1 to 7, and R″ is hydrogen or a N-protecting group such as N-Boc, and O/NH indicates that the branch is linked to the polymer backbone by an amide linkage formed with an amine group on the or an ester linkage formed with a hydroxy group on the polymer backbone. In a preferred embodiment, a polymer backbone with amine residues is amidated with an O-hydroxylamine spacer.

The present invention is also directed to O-hydroxylamine esterified or amidated cyclodextrins according to the formula

a) [NHR″O—(S)_{q}—CO—O/NH]ₙ-cyclodextrin (IId)

or specifically

b) [NHR″O—(CH₂)_{q2}—CO—O/NH]ₙ-cyclodextrin (IId′)

wherein the symbols R″, S, q, q2 and n have the meaning given in the formula (IIa).

Preferred cyclodextrins include alfa, -beta- and gamma-cyclodextrins, beta- or gamma-cyclodextrin being preferred.

Such conjugates are preferably made by esterifying N—BOC aminooxyacetic acid with cyclodextrin in dry pyridin with an excess of N—BOC-aminooxyacetic acid and activators, i.e. reagents activating the esterification reactions. Preferred activating reagents includes HBTU and diisopropylamine.

The present invention is also directed to the use of intermediate products according to the formula

Carb-NR′—O—(S)_{q}-(z′)ᵣ (III)

wherein Carb is carbohydrate, such as an oligo- or monosaccharide, R′ is hydrogen or a further bond to Carb, S, q and r have the meanings given in the formula II and z′ is a group that can react with the polymer, such as the polysaccharide and/or carbohydrate backbone structure PO to form the group z, where z is as defined, for conjugation with a polymer, such as a polysaccharide and/or carbohydrate backbone structure, preferably with an carbohydrate backbone structure, and more preferably a chitosan oligomer backbone structure.

Such conjugate structures can have the formulas

Carb-NR′—O—(S)_{q}—COR′″ (IIIa)

or specifically

Carb-NR′—O—(CH₂)_{q2}—COR′″ (IIIa′)

wherein the symbols S, q, q2 and n have the meaning given in the formula (IIa), and R′ is the same as in the formula (M), R′″ is OH or a carboxylic acid activating conjugate, preferably a succinimide ester.

### Biorecognition Molecules

Preferred biorecognition molecules are those which occur on cell surfaces as components of glycoproteins, glycolipids or proteoglycans, as well as any desired segments thereof. Particularly preferred biorecognition molecules are those composed of monosaccharides which also occur in the human body, such as glucose, N-acetylglucosamine, galactose, N-acetylgalactosamine, mannose, fucose, N-acetylneuraminic acid and glucuronic acid.

The monosaccharide units forming the biorecognition molecule may be identical or different. In addition, the stereochemistry of the glycosidic linkage (axial or equatorial, or .alpha. or .beta.) of the individual monosaccharide units may be identical or different.

The biorecognition molecule can be composed, for example, of the following sugar residues:

Gal.beta.1-4GlcNAc-;
Gal.beta.1-3GlcNAc-;
SA.alpha.2-6Gal.beta.1-4GlcNAc-;
SA.alpha.2-3Gal.beta.1-4GlcNAc-;
SA.alpha.2-3Gal.beta.1-3GlcNAc-;
Gal.beta.1-4(Fuc.alpha.1-3)GlcNAc-;
Gal.beta.1-4(Fuc.alpha.1-3)GlcNAc-;
Gal.beta.1-3(Fuc.alpha.1-3)GlcNAc-;
SA.alpha.2-3Gal.beta.1-3(Fuc.alpha.1-4)GlcNAc-;
SA.alpha.2-3Gal.beta.1-4(Fuc.alpha.1-3)GlcNAc-;
Gal.beta.1-4GlcNAc.beta.1-4GlcNAc-;
Gal.beta.1-3GlcNAc.beta.1-4GlcNAc-;
SA.alpha.2-6Gal.beta.1-4GlcNAc.beta.1-4GlcNAc-;
SA.alpha.2-3Gal.beta.1-4GlcNAc.beta.1-4GlcNAc-;
SA.alpha.2-3Gal.beta.1-3GlcNAc.beta.1-4GlcNAc-;
Gal.beta.1-4(Fuc.alpha.1-3)GlcNAc.beta.1-4GlcNAc-;
Gal.beta.1-3(Fuc.alpha.1-4)GlcNAc.beta.1-4GlcNAc-;
SA.alpha.2-3Gal.beta.1-3(Fuc.alpha.1-4)GlcNAc.beta.1-4GlcNAc-;
SA.alpha.2-3Gal.beta.1-4(Fuc.alpha.1-3)GlcNAc.beta.1-4GlcNAc-;
SA.alpha.2-3Gal.beta.1-3(Fuc.alpha.1-4)GlcNAc.beta.1-4Gal-;
SA.alpha.2-3Gal.beta.1-4(Fuc.alpha.1-3)GlcNAc.beta.1-4Gal-;
SA.alpha.2-3Gal.beta.1-4GlcNAc.beta.1-3Gal.beta.1-4(Fuc.alpha.1-3)GlcNAc;
SA.alpha.2-6Gal.beta.1-4GlcNAc.beta.1-3Gal.beta.1-4(Fuc.alpha.1-3)GlcNAc;
SA.alpha.2-3Gal.beta.1-4(Fuc.alpha.1-3)GlcNAc.beta.1-3Gal.beta.1-4Glc;
SA.alpha.2-6Gal.beta.1-4(Fuc.alpha.1-3)GlcNAc.beta.1-3Gal.beta.1-4Glc;
SA.alpha.2-3Gal.beta.1-4GlcNAc.beta.1-3Gal.beta.1-4(Fuc.alpha.1-3)GlcNAc1-3 Gal.beta.1-4Glc;
SA.alpha.2-6Gal.beta.1-4GlcNAc.beta.1-3Gal.beta.1-4(Fuc.alpha.1-3)GlcNAc1-3 Gal.beta.1-4Glc;
SA.alpha.2-3Gal.beta.1-4(Fuc.alpha.1-3)GlcNAc.beta.1-3Gal.beta.1-4(Fuc.alpha.1-3)GlcNAc.beta.1-3Gal.beta.1-4Glc;
SA.alpha.2-6Gal.beta.1-4(Fuc.alpha.1-3)GlcNAc.beta.1-3Gal.beta.1-4(Fuc.alpha.1-3)GlcNAc1-3Gal.beta.1-4Glc;
SA.alpha.2-3Gal.beta.1-4(Fuc.alpha.1-3)GlcNAc.beta.1-3Gal.beta.1-4(Fuc.alpha.1-3)Glc;
SA.alpha.2-6Gal.beta.1-4(Fuc.alpha.1-3)GlcNAc.beta.1-3Gal.beta.1-4(Fuc.alpha.1-3)Glc;
SA.alpha.2-3Gal.beta.1-4GlcNAc.beta.1-3Gal.beta.1-4(Fuc.alpha.1-3)GlcNAc.beta.1-3Gal.beta.1-4(Fuc.alpha.1-3)Glc;
SA.alpha.2-6Gal.beta.1-4GlcNAc.beta.1-3Gal.beta.1-4(Fuc.alpha.1-3)GlcNAc.beta.2-3Gal.beta.1-4(Fuc.alpha.1-3Glc;
SA.alpha.2-3Gal.beta.1-4(Fuc.alpha.1-3)GlcNAc.beta.1-3Gal.beta.1-4(Fuc.alpha.1-3)GlcNAc.beta.1-3Gal.beta.1-4(Fuc.alpha.1-3)Glc;
SA.alpha.2-6Gal.beta.1-4(Fuc.alpha.1-3)GlcNAc.beta.1-3Gal.beta.1-4(Fuc.alpha.1-3)GlcNAc.beta.1-3Gal.beta.1-4(Fuc.alpha.1-3)Glc;
SA.alpha.2-3Gal.beta.1-3(Fuc.alpha.1-4)GlcNAc;
SA.alpha.2-6Gal.beta.1-3(Fuc.alpha.1-4)GlcNAc;
SA.alpha.2-3Gal.beta.1-3GlcNAc.beta.1-4Gal.beta.1-4(Fuc.alpha.1-3)GlcNAc;
SA.alpha.2-6Gal.beta.1-3GlcNAc.beta.1-4Gal.beta.1-4(Fuc.alpha.1-3)GlcNAc;
SA.alpha.2-3Gal.beta.1-3(Fuc.alpha.1-4)GlcNAc.beta.1-3Gal.beta.1-4(Fuc.alpha.1-3)GlcNAc;
SA.alpha.2-6Gal.beta.1-3(Fuc.alpha.1-4)GlcNAc.beta.1-3Gal.beta.1-4(Fuc.alpha.1-3)GlcNAc;
SA.alpha.2-3Gal.beta.1-3(Fuc.alpha.1-4)GlcNAc.beta.1-3Gal.beta.1-4Glc;
SA.alpha.2-6Gal.beta.1-3(Fuc.alpha.1-4)GlcNAc.beta.1-3Gal.beta.1-4Glc;
SA.alpha.2-3Gal.beta.1-3GlcNAc.beta.1-4Gal.beta.1-4(Fuc.alpha.1-3)GlcNAc.beta.1-3Gal.beta.1-4Glc;
SA.alpha.2-6Gal.beta.1-3GlcNAc.beta.1-4Gal.beta.1-4(Fuc.alpha.1-3)GlcNAc.beta.1-3Gal.beta.1-4Glc;
SA.alpha.2-3Gal.beta.1-3(Fuc.alpha.1-4)GlcNAc.beta.1-3Gal.beta.1-4(Fuc.alpha.1-3)GlcNAc.beta.1-3Gal.beta.1-4Glc;
SA.alpha.2-6Gal.beta.1-3(Fuc.alpha.1-4)GlcNAc.beta.1-3Gal.beta.1-4(Fuc.alpha.1-3)GlcNAc.beta.1-3Gal.beta.1-4Glc;
SA.alpha.2-3Gal.beta.1-3(Fuc.alpha.1-4)GlcNAc.beta.1-3Gal.beta.1-4(Fuc.alpha.1-3)Glc;
SA.alpha.2-6Gal.beta.1-3(Fuc.alpha.1-4)GlcNAc.beta.1-3Gal.beta.1-4(Fuc.alpha.1-3)Glc;
SA.alpha.2-3Gal.beta.1-3GlcNAc.beta.1-4Gal.beta.1-4(Fuc.alpha.1-3)GlcNAc.beta.1-3Gal.beta.1-4(Fuc.alpha.1-3)Glc;
SA.alpha.2-6Gal.beta.1-3GlcNAc.beta.1-4Gal.beta.1-4(Fuc.alpha.1-3)GlcNAc.beta.1-3Gal.beta.1-4(Fuc.alpha.1-3)Glc;
SA.alpha.2-3Gal.beta.1-3(Fuc.alpha.1-4)GlcNAc.beta.1-3Gal.beta.1-4(Fuc.alpha.1-3)GlcNAc.beta.1-3Gal.beta.1-4(Fuc.alpha.1-3)Glc;
SA.alpha.2-6Gal.beta.1-3(Fuc.alpha.1-4)GlcNAc.beta.1-3Gal.beta.1-4(Fuc.alpha.1-3)GlcNAc.beta.1-3Gal.beta.1-4(Fuc.alpha.1-3)Glc;
SA.alpha.2-3Gal.beta.1-4GlcNAc.beta.1-3Gal.beta.1-3(Fuc.alpha.1-4)GlcNAc;
SA.alpha.2-6Gal.beta.1-4GlcNAc.beta.1-3Gal.beta.1-3(Fuc.alpha.1-4)GlcNAc;
SA.alpha.2-3Gal.beta.1-4(Fuc.alpha.1-3)GlcNAc.beta.1-3Gal.beta.1-3(Fuc.alpha.1-4)GlcNAc;
SA.alpha.2-6Gal.beta.1-4(Fuc.alpha.1-3)GlcNAc.beta.1-3Gal.beta.1-3(Fuc.alpha.1-4)GlcNAc;
SA.alpha.2-3Gal.beta.1-4GlcNAc.beta.1-3Gal.beta.1-3(Fuc.alpha.1-4)GalcNAc.beta.1-3Gal.beta.1-4Glc;
SA.alpha.2-6Gal.beta.1-4GlcNAc.beta.1-3(Fuc.alpha.1-4)GalcNAc.beta.1-3Gal.beta.1-4Glc;
SA.alpha.2-3Gal.beta.1-4(Fuc.alpha.1-3)GlcNAc.beta.1-3Gal.beta.1-3(Fuc.alpha.1-4)GlcNAc.beta.1-3Gal.beta.1-4Glc;
SA.alpha.2-6Gal.beta.1-4(Fuc.alpha.1-3)GlcNAc.beta.1-3Gal.beta.1-3(Fuc.alpha.1-4)GlcNAc.beta.1-3Gal.beta.1-4Glc;
SA.alpha.2-3Gal.beta.1-4(Fuc.alpha.1-3)GlcNAc.beta.1-3Gal.beta.1-3(Fuc.alpha.1-4)Glc;
SA.alpha.2-6Gal.beta.1-4(Fuc.alpha.1-3)GlcNAc.beta.1-3Gal.beta.1-3(Fuc.alpha.1-4)Glc;
SA.alpha.2-3Gal.beta.1-4GlcNAc.beta.1-3Gal.beta.1-3(Fuc.alpha.1-4)GlcNAc.beta.1-3Gal.beta.1-4(Fuc.alpha.1-3)Glc;
SA.alpha.2-6Gal.beta.1-4GlcNAc.beta.1-3Gal.beta.1-3(Fuc.alpha.1-4)GlcNAc.beta.1-3Gal.beta.1-4(Fuc.alpha.1-3)Glc;
SA.alpha.2-3Gal.beta.1-4(Fuc.alpha.1-3)GlcNAc.beta.1-3Gal.beta.1-3(Fuc.alpha.1-4)GlcNAc.beta.1-3Gal.beta.1-4(Fuc.alpha.1-3)Glc;
SA.alpha.2-6Gal.beta.1-4(Fuc.alpha.1-3)GlcNAc.beta.1-3Gal.beta.1-3(Fuc.alpha.1-4)GlcNAc.beta.1-3Gal.beta.1-4(Fuc.alpha.1-3)Glc;
[GlcNAc.beta.-1-3Gal.beta.1-4].sub.n GlcNAc-, where n is a number from the series from 1 to 8;
[GlcNAc.beta.-1-3Gal.beta.1-4].sub.n GlcNAc.beta.1-4GlcNAc-, where n is a number from the series from 1 to 8;
Gal.beta.-1-4-[GlcNAc.beta.1-3Gal.beta.1-4].sub.n GlcNAc-, where n is a number from the series from 1 to 8;
Gal.beta.-1-3-[GlcNAc.beta.1-3Gal.beta.1-4].sub.n GlcNAc-, where n is a number from the series from 1 to 8;
SA.alpha.2-6Gal.beta.1-4[GlcNAc.beta.1-3Gal.beta.1-4].sub.n GlcNAc-, where n is a number from the series from 1 to 8;
SA.alpha.2-3Gal.beta.1-4[GlcNAc.beta.1-3Gal.beta.1-4].sub.n GlcNAc-, where n is a number from the series from 1 to 8;
SA.alpha.2-3Gal.beta.1-3[GlcNAc.beta.1-3Gal.beta.1-4].sub.n GlcNAc-, where n is a number from the series from 1 to 4;
Gal.beta.1-4(Fuc.alpha.1-3)GlcNAc.beta.1-3[Gal.beta.1-4(Fuc.alpha.1-3).sub.m-GlcNAc.beta.1-3].sub.n-Gal.beta.1-4GlcNAc-, where m is a number from the series from 0 to 1 and where n is a number from the series from 1 to 4;
Gal.beta.1-3(Fuc.alpha.1-4)GlcNAc.beta.1-3[Gal.beta.1-4(Fuc.alpha.1-3).sub.m-GlcNAc.beta.1-3].sub.n-Gal.beta.1-4GlcNAc-, where m is a number from the series from 0 to 1 and where n is a number from the series from 1 to 8;
SA.alpha.2-3Gal.beta.1-3(Fuc.alpha.1-4)GlcNAc.beta.1-3-[Gal.beta.1-4GlcNAc.beta.-3].sub.n-Gal.beta.1-4GlcNA-, where n is a number from the series from 1 to 8;
SA.alpha.2-3Gal.beta.1-4(Fuc.alpha.1-3)GlcNAc.beta.1-3[Gal.beta.1-4(Fuc.alpha.1-3).sub.m-GlcNAc.beta.-1-3].sub.n-Gal.beta.1-4GlcNA-, where m is a number from the series from 0 to 1 and where n is a number from the series from 1 to 2;
Gal.beta.-1-4[GlcNAc.beta.1-3Gal.beta.1-4].sub.n GlcNAc.beta.1-4GlcNAc-, where n is a number from the series from 1 to 8;
Gal.beta.-1-3[GlcNAc.beta.1-3Gal.beta.1-4].sub.n GlcNAc.beta.1-4GlcNAc-, where n is a number from the series from 1 to 8;
SA.alpha.2-6Gal.beta.1-4[GlcNAc.beta.1-3Gal.beta.1-4].sub.n GlcNAc.beta.1-4GlcNAc-, where n is a number from the series from 1 to 8;
SA.alpha.2-3Gal.beta.1-4[GlcNAc.beta.1-3Gal.beta.1-4].sub.n GlcNAc.beta.1-4GlcNAc-, where n is a number from the series from 1 to 8;
SA.alpha.2-3Gal.beta.1-3[GlcNAc.beta.1-3Gal.beta.1-4].sub.n GlcNAc.beta.1-4GlcNAc-, where n is a number from the series from 1 to 8;
Gal.beta.1-4(Fuc.alpha.1-3)GlcNAc.beta.1-3[Gal.beta.1-4(Fuc.alpha.1-3).sub.m GlcNAc.beta.1-3].sub.n-
Gal.beta.1-4GlcNAc.beta.1-4GlcNAc-, where m is a number from the series from 0 to 1 and where n is a number from the series from 1 to 4;
Gal.beta.1-3(Fuc.alpha.1-4)GlcNAc.beta.1-3[Gal.beta.1-4(Fuc.alpha.1-3).sub. m GlcNAc.beta.1-3].sub.n-
Gal.beta.1-4GlcNAc.beta.1-4GlcNAc-, where m is a number from the series from 0 to 1 and where n is a number from the series from 1 to 4;
SA.alpha.2-3Gal.beta.1-3(Fuc.alpha.1-4)GlcNAc.beta.1-3[Gal.beta.1-4GlcNAc.beta.1-3].sub.n-
Gal.beta.1-4GlcNAc.beta.1-4GlcNAc-, where n is a number from the series from 1 to 6;
(GlcNAc.beta.1-3Gal.beta.1-4).sub.n GlcNAc.beta.1-3Gal, where n is a number from the series from 1 to 8;
SA.alpha.2-6Gal-;
SA.alpha.2-6Gal.beta.1-4(GlcNAc.beta.1-3Gal.beta.1-4).sub.n GlcNAc.beta.1-3Gal, where n is a number from the series from 1 to 10;
SA.alpha.2-3Gal-; and
SA.alpha.2-3Gal.beta.1-4(GlcNAc.beta.1-3Gal.beta.1-4).sub.n GlcNAc.beta.1-3Gal, where n is a number from the series from 1 to 10.

Examples of preferred embodiments of the biorecognition molecules are sialyl-Lewis X, sialyl-Lewis A, VIM-2 and the following blood-group determinants Lewis A, B, X, Y and Z type.sup.1, A type.sup.2, B type.sup.1, B type.sup.2 and H type.sup.1, H type.sup.2. R. U. Lemieux, Chem. Soc. Rev. 7:423 (1978) and 18:347 (1989).

Examples of most preferred embodiments of the biorecognition molecules are sialyl-Lewis X, sialyl-Lewis A or VIM-2. The formula of sialyl-Lewis X is NeuNAc.alpha.2-3Gal.beta.1-4(Fuc.alpha.1-3)GlcNAc and of sialyl-Lewis A NeuNAc.alpha.2-3-Gal.beta.1-3(Fuc.alpha.1-4)GlcNAc. The formula of VIM-2 is NeuNAc.alpha.2-3Gal.beta.1-4GlcNAc.beta.1-3Gal.beta.1-4(Fuc.alpha.1-3)GlcNAc.

The carbohydrate biorecognizable molecules include counter-receptors for various cell or tissue surface receptors. The receptors include various lectin proteins and other carbohydrate recognizing protein molecules such as acidic carbohydrate binding proteins, e.g. glycosaminoglycan binding proteins or lectins, glycosidases, glycosyltransferases, transglycosylases and glycosidases. The receptors for carbohydrates also include carbohydrate epitopes participating in carbohydrate interactions on cell surfaces.

The present invention is especially directed to the inhibition of binding of pathogenic bacteria, viruses or toxins to cell surface receptors. Such pathogenic bacteria or toxins thereof include for example the gastric pathogen Helicobacter pylori, diarrhea causing Escherichia coli, E. coli causing urinary tract infections, Salmonella species, Vibrio species, Campylobacter, pneumonia causing bacteria including Streptococcus species, Haemophilus species, Pseudomonas species or Klebsiella species. A number of polyvalent carbohydrate constructs have been produced for inhibition of bacteria or toxins, e.g. Synsorb Biotech's oligosaccharide linked by a long spacer to a silica polymer.

The distances between the biorecognition molecules in a polyvalent or oligovalent construct can be optimised for various receptors. At an optimal distance the spacing sequence between the biorecognition molecules allows simultaneous binding to the receptors while there is no or little extraneous spacing which could cause entropic penalty for the binding. The optimal distances can be determined from crystal structures of multidomain receptors or models of cell membranes. On cell membranes the receptors may be able to cluster or may be more fixed on certain positions by cytoskeleton. For these cases constructs of polymeric or oligomeric biorecognition molecules at optimal distance are constructed. Moreover, the present invention aims for sterically effective representation of the biorecognition molecules. This means mimicking the natural representation of the biorecognition molecules.

Specifically, in accordance with the formula presented above, to the defined carrier structure, i.e. to an carbohydrate backbone structure as defined, or a mixture thereof, Bio-(y)ₚ-(S)_{q}-(z)ᵣ- structures as defined are covalently linked. Preferably the groups Bio-(y)ₚ-(S)_{q}-(z)ᵣ- are linked to different monosaccharide units on an oligosaccharide carrier, meaning that each monomer unit carries at the most one biorecognition structure. In a cyclodextrin backbone, the substitution is preferably selectively at the primary hydroxyl in the 6-position of the monosaccharide, and in a chondroitin mer in at the carboxylic acid in the 6-position of the monosaccharide.

According to a preferred embodiment, in an oligosaccharide structure as defined the conjugation ratio is such that there is on an average 2 to 10, preferably 3 to 7, and most preferably 4-6 biorecognizable groups for every 10 monomer units, that is the conjugation ratio is high, being on an average 0.2-1, with respect to the monomer unit.

In a preferred embodiment, the Bio-structure is a reducing carbohydrate, preferably an oligosaccharide or a monosaccharide, linked through a hydroxylamine glycosidic linkage. The hydroxylamine glycosidic linkage is formed so that the terminal amine of an O-hydroxylamine structure reacts selectively with the reducing end aldehyde/hemiacetal or ketone/hemiketal structures of the oligosaccharide or the monosaccharide.

In a preferred embodiment, the Bio-structure is a reducing carbohydrate, preferably an oligosaccharide or a monosaccharide, attached to the spacer at Cl at its reducing end.

Preferred monosaccharides to be conjugated to polyvalent forms include D-, and L-hexoses and pentoses and sialic acids. The hexoses may be modified to natural hexosamines or N-acetylhexosamines or hexuronic acids. The preferred monosaccharides to be coupled to polyvalent form include GlcN, GlcNAc, GlcA, Glc, GalNAc, GalN, Gal, IdoA, GalA, Xyl, Man, sialic acid, Fuc, more preferably GlcN, GlcNAc, GlcA, Glc, GalNAc, Gal, Gala, Man, Xyl, IdoA, sialic acid, and Fuc.

The preferred oligosaccharides preferably comprise the Helicobacter pylori inhibiting oligosaccharide sequences developed by the inventors and collaborators including oligosaccharide sequences according to the formula:

[Hex1(A)_{q1}(NAc)ᵣ₁y3]ₛGal(NAc)ᵣ₂β4Glc(A)_{q2}(NAc)ᵣ₃

wherein q1, q2, r1, r2, r3 and s are each independently 0 or 1, and Hex1 is a hexose structure, preferably galactose (Gal) or glucose (Glc) or mannose (Man), most preferably Gal or Glc, which may be further modified by the A and/or NAc groups; y is either alpha or beta indicating the anomeric structure of the terminal monosaccharide residue, as well as analogs or derivatives of said oligosaccharide sequence for binding or inhibiting Helicobacter pylori.

In a preferred embodiment, the invention is directed to the synthesis of polyvalent conjugates from oligosaccharide type epitopes ranging from disaccharide substances to pentasaccharide substances, more preferably from disaccharide substances to tetrasaccharide substances. According to a further embodiment the present invention is directed to the synthesis of polyvalent conjugates from trisaccharide substances and from tetrasaccharide substances.

Preferably the oligosaccharide sequences include Lacto-N-neotetraose (LNnT) Galβ4GlcNAcβ3Galβ4Glc and its elongated variant GlcNAcβ3Galβ4GlcNAcβ3Galβ4Glc. These oligosaccharide sequences are described in WO2004/065400.

The preferred oligosaccharide sequences for inhibition of pathogens, especially H. pylori, further include oligosaccharides with the terminal sequence Galβ3GlcNAc, more preferably Galβ3GlcNAcβ3Galβ4Glc (WO0143751), Lewis b structures, Fucα2Galβ3(Fucα4)GlcNAc (WO9747646) and H-antigen comprising oligosaccharide sequences, with some activity towards H. pylori and/or other pathogenic bacteria, Fucα2Galβ3, preferably Fucα2Galβ4GlcNAc, Fucα2Galβ3GlcNAc, Fucα2Galβ4GlcNAcβ3Galβ4Glc, and Fucα2Galβ3GlcNAcβ3Galβ4Glc; oligosaccharides with terminal sialyl-lactosamine, Neu5Acα3/6Galβ4GlcNAc, Neu5Acα3/6Galβ3GlcNAc, sialyl-lactoses Neu5Acα3/6Galβ4Glc, or sialyl-Lewis antigens sialyl Lewis a, Neu5Acα3Galβ3(Fucα4)GlcNAc, and sialyl-Lewis x, Neu5Acα3Galβ4(Fucα3)GlcNAc, the Neu5Acα3-structures are known to bind H. pylori (WO0056343). The present invention is further directed to substances containing sialyl-lactoses and sialyl-lactosamines and elongated oligosaccharide forms thereof such as Neu5Acα3/6Galβ4GlcNAcβ3Galβ4Glc in polyvalent and divalent forms.

A preferred conjugate with reducing carbohydrate has the formula

[Carb-NR′—O—(S)_{q}-(z)ᵣ]ₙPO (Ib)

wherein PO is a poly- or oligosaccharide, Carb is the reducing carbohydrate coupled from the reducing end by a hydroxylamine linkage —NR′—O— wherein R′ is H or forms a second bond to the reducing end of Carb, and S, z, q, r and n have the meaning given in the formula (I).

In a preferred embodiment q is 1 and r is 1. In a further preferred embodiment the z-group is an amide group formed from a carboxylic acid with an amine group of the PO-structure, or the z-group is an ester group formed from a carboxylic acid with a hydroxy group on the PO-structure, thus forming the structures

[Carb-NR′—O—(S)_{q}—CO—O/NH]ₙ—PO (Ic)

wherein the symbols have the meaning given above in the formula (Ib).

The spacer structure is preferably a lower alkyl-structure. In a preferred embodiment S is —CH₂—. A preferred spacer reagent is thus aminooxy acetic acid forming structures

[Carb-NR′—O—(CH₂)_{q}—CO—O/NH]ₙ—PO (Id)

wherein the symbols have the meaning given above in the formula (Ib).

An another preferred conjugate with reducing carbohydrate has the formula

[Carb-NR′—(S)_{q}—CO—O/NH]ₙ—PO (2a)

wherein PO is a poly- or oligosaccharide, Carb is the reducing carbohydrate coupled from the reducing end by a amide or a amine linkage to —NR′ wherein R′ is H or forms a second bond to the reducing end of Carb, and S, q, and n have the meaning given in the formula (I).

An another preferred conjugate with reducing carbohydrate has the formula

[Carb-NH—CO]ₙ—PO (2b)

wherein PO is a poly- or oligosaccharide, Carb is the reducing carbohydrate converted to a glycosylamine and coupled by an amide linkage to a C═O unit originating from a carboxylic acid group of PO, and n has the meaning given in the formula (I).

An another preferred conjugate with a carbohydrate has the formula

[Carb-CO—NH]ₙ—PO (2c)

wherein PO is a poly- or oligosaccharide, Carb is a carbohydrate carrying a carboxylic acid group and coupled by a amide linkage to a NH unit originating from an amine group of PO, and n has the meaning given in the formula (I).

### Chemoselective Ligation Groups

The chemoselective ligation group y and/or z is a chemical group allowing the coupling of the Bio-group and the backbone PO to a spacer group, with or without using protecting groups or catalytic or activator reagents in the coupling reaction. Examples of chemoselective ligation groups y and z which may be present include the hydrazino group —N—NH— or —N—NR₁—, the ester group —C(═O)—O—, the keto group —C(═O)—, the amide group —C(═O)—NH—, the O-hydroxylamine residue —O—NH— or —O—N═, —O—, —S—, —NH—, —NR₁—, etc., wherein R₁ is H or a lower alkyl group, preferably containing up to 6 carbon atoms, etc. A preferred chemoselective ligation group is the ester group —C(═O)—O— formed with a hydroxy group, and the amide group —C(═O)—NH— formed with an amine group on the PO or Bio group, respectively. Preferably p, q, and r are 1. If q is 0, then preferably one of p and r is 0, that is, there is one linking group between Bio and PO.

The chemoselective ligation groups are divided into primary chemoselective groups which are active for specific coupling in water solutions without any activation chemicals. In a specific embodiment the primary chemoselective group O-hydroxylamine is replaced by an analogous reagent capable of specific conjugation to Bio or PO. The alternative primary chemoselective groups include one of the pair: hydrazide group and aldehyde/or ketone, or phosphine group and azide group which are reactive in the Staudinger reaction to form an amide as described in U.S. Pat. No. 6,570,040, thiol group and maleimide group which form covalent linkages. Before the conjugation, the chemosective groups are in active forms and in conjugates the chemoselective groups are in conjugated forms, the active forms are specified by markings such as y′ and z′ in the present description.

Secondary chemoselective groups may be more useful to react even in non-water solution and in the presence of an activating chemical. The preferred secondary chemoselective group includes one of the pair carboxylic acid and hydroxyls to form an ester bond and the pair carboxylic acid and amine to form amides. The secondary chemoselective groups allow chemoselective ligation of hydroxyl, amine or carboxylic acid groups on carbohydrates without the need of protecting the carbohydrate. The reaction to form esters needs to be controlled to avoid polymerisation when the carboxylic acid group is on the carbohydrate.

The present invention is preferably directed to the use of chemoselective ligations of natural hydroxyl-, carboxylic acid, amine, and aldehyde or ketone groups present on biomolecules. The use of natural carbohydrate structures reduces the need for introducing reactive groups for alternative chemistries, such as azide, phosphine, thiol, or maleimide, in a carbohydrate.

In a specific embodiment, the carboxylic acid may be used in the form of activated ester such as succinimide ester or sulfosuccinimide ester, paranitrophenylester, or pentafluorophenol ester, as a primary chemoselective group or alternative primary chemoselctive group to form amide or ester bonds. The stabilities of the activated esters limit the effectivity of the reactions in water solutions.

The conjugates according to the present invention may include two primary chemoselective groups or one primary and one secondary chemoselective group. In a specific separate embodiment using ester bond, even two secondary chemoselective groups are used.

In a specific separate embodiment, at least one of the groups y and z is an ester group. The ester group is preferred as biodegradable structure. If the ester linkage is degraded, the carbohydrate backbone remains intact. The ester group is preferred as it can be formed directly between natural carbohydrate structures from a carbohydrate containing a carboxylic acid, such as an uronic acid residue or for example a carboxymethyl derivative of a carbohydrate, to a hydroxyl of a second carbohydrate. A carboxylic acid containing spacer can also be coupled effectively to an oligosaccharide carbohydrate backbone. The use of ester bonds is possible because the inventors found that some of the ester conjugation reactions for the molecules according to the invention can be carried out in pyridin.

The chemoselective ligation group is selected so that possible functional groups present on the carbohydrate backbone or on a Bio-group will not disturb the conjugation process. Chemoselective ligation is used to perform a chemical coupling between the non-protected carbohydrate backbone and the biorecognition group preferably in water solutions. Preferably at least one of the chemoselective reaction groups is a chemoselective group which is reactive without the need for activation of the linking reaction.

According to a specific embodiment, an activated ester of a carboxylic acid, such as a succinimide ester is used. Preferably the activated ester is an ester of an uronic acid such as a methyl ester of an uronic acid. Alternatively the chemoselective ligation group is a labile chemical linkage which may be specifically directed to the carbohydrate structures according to the invention. The labile chemical linkages may be selectively released during biodegradation processes and therefore they are less likely to accumulate in the body or cause adverse immune reactions. A preferred labile chemical linkage is an ester linkage.

The ester linkage has chemoselective activity and can e.g. be formed when a carboxylic acid of the Bio group or Bio-(y)ₚ-(S)_{q}-(z)ₙ- group is linked to a polymer comprising secondary and primary alcohol groups.

### Spacer Groups

According to an embodiment of the invention the spacer group, when present, is preferably selected from a straight or branched alkylene group with 1 to 10, preferably 1 to 6 carbon atoms, or a straight or branched alkenylene or alkynylene group with 2 to 10, or 2 to 6 carbon atoms. Preferably such group is a methylene, ethylene or propylene group. In the spacer group one or more of the chain members can be replaced by —NH—, —O—, —S—, —S—S—, ═N—O—, an amide group —C(O)—NH— or —NH—C(O)—, an ester group —C(O)O— or —O—C(O)—, or —CHR₂, where R₂ is an alkyl or alkoxy group of 1 to 6, preferably 1 to 3 carbon atoms, or —COOH. Preferably a group replacing a chain member is —NH—, —O—, an amide or an ester group.

### Conjugates Comprising Uronic Acid and or Amine Structures

The present invention revealed novel useful carbohydrate conjugates produced from carbonyl groups of natural carbohydrates or carbonyl groups, which can be synthesized to the natural carbohydrates and or amine groups on carbohydrates, preferably amine groups of natural carbohydrates. The invention is further especially directed to specific production methods of the conjugates according to the invention and useful middle products of the process.

The carbohydrates according to the present invention preferably comprise sugar residues with six membered rings referred here as pyranoses, and preferably being actual pyranose residues or analogs or derivatives thereof and/or with five membered ring structures referred as furanoses and preferably being actual furanose residues or analogs or derivatives thereof.

The analogs and derivatives are regular known analogs and/or derivatives of carbohydrates and preferably includes deoxy and/or anhydro and/or amino structures and derivatives threof. Preferred pyranoses are hexoses and furanoses pentoses, which are preferably naturally occurring monosaccharide residues more preferably naturally occurring human or animal monosaccharide residues.

In a preferred embodiment at least one carbohydrate and preferably both first and second carbohydrates are oligosaccharides or polysaccharides.

### Valency of the Conjugates

### Monovalent Carbohydrate Conjugates

In a preferred embodiment the invention is directed to carbohydrate-carbohydrate conjugates according to the invention, wherein one of the first and one of the second carbohydrate is linked to each other as monovalent carbohydrate conjugate.

### Oligovalent or Polyvalent Conjugates

In a preferred embodiment the invention is directed to carbohydrate-carbohydrate conjugates according to the invention, wherein one of the carbohydrates is oligovalent or polyvalent carrier (the first or second carbohydrate) and linked to two or more of the other (first or second) carbohydrate as oligovalent or polycarbohydrate conjugate. Oligovalent conjugates comprise 2 to 10 carbohydrates and polyvalents more than 10.

More preferably, one carbohydrate is a polyvalent carrier modified by at least one, more preferably by at least two of the other carbohydrates and even more preferably by at least three and even more preferably by at least four of the other carbohydrates, and in a separate embodiment preferred oligovalent oligomers from 2-10, preferably 3-10, or 3-8, more preferably 4-0 or 4-8 of the other carbohydrates or polymer comprising more than 10 of the other carbohydrates, but in a preferred embodiment less than 10 million and in separate embodiment 10 to million or 10 to 100 000 of the other carbohydrates, and in another preferred embodiment 10-1000 or even more preferably smaller polyvalent conjugates comprising 10-100 of the other carbohydrates.

It is realized that effective controlled production of oligovalent and/or polyvalent conjugates, even with specific substitution levels, is especially challenging when non-protected carbohydrates is used and there is also steric challenges in the effective construction methods.

### Additional Carriers

The conjugates of the first and second carbohydrates according to the invention are in a preferred embodiment linked to an additional monovalent or polyvalent carrier, more preferably monovalent carbohydrate-carbohydrate conjugates are linked to a polyvalent carrier. The additional polyvalent carrier is preferably a water soluble polymeric carrier, in a preferred embodiment a natural polyvalent carrier such as a protein.

In a preferred embodiment an oligosaccharide or polysaccharide, preferably a glycosaminoglycan or fragment thereof or a cyclodextrin is linked to a carbohydrate chain(s) linked to a protein. The glycan chains are preferably natural N-glycans or O-glycans or glycoasaminoglycans of a protein, more preferably N- or O-glycans. Preferred method to modify glycans of proteins to comprise useful linking groups for present methods includes e.g.:

1) enzymatic transfer of reactive groups as previously described in copending applications of the present applicant and part of the inventors or in patent application and publications of Pradman Qasba and colleagues using modified galactosyltransferase and other transferases or by methods of Neose,
2) enzymatic oxidation of carbohydrates by galactoseoxidase or by possible other hexose oxidases as described in HES-conjugation patents of Kabi-Frensenius and others
3) chemical oxidation such as periodic acid oxidation, when reasonably tolerated by the protein and its application.

### The Structures of Preferred Carbohydrate Carbohydrate Conjugates

The invention is in a preferred embodiment directed to novel biorecognition conjugates derived from

a first carbohydrate, preferably comprising at least one monosaccharide, or at least one oligosaccharide (including disaccharides and larger oligosaccharides from trimer to decamers) or at least one polysaccharide residue, comprising
i) at least one monosaccharide residue comprising a carbonyl group, preferably a reducing end carbonyl group or a carbonyl group linked to a furanose (or five membered) or pyranose (or six membered ring, more preferably a carbonyl group linked to a furanose or pyranose ring, and in a preferred embodiment preferably a pyranose structure of the carbohydrate
   and/or
ii) at least one monosaccharide residue comprising an amine group linked to a furanose or pyranose ring. The amine is a primary amine, glycosylamine or secondary amine, preferably a primary amine, preferably on a 2-position of furanose or pyranose ring.
   and
   a second carbohydrate, preferably comprising at least one monosaccharide, or at least one oligosaccharide (including disaccharides and larger oligosaccharides from trimer to decamers) or at least one polysaccharide residue, comprising
a) at least one monosaccharide residue comprising a carbonyl group, preferably a reducing end carbonyl group or a carbonyl group linked to a furanose or pyranose ring, more preferably a carbonyl group linked to a furanose or pyranose ring, and in a preferred embodiment preferably a pyranose structure of the carbohydrate and/or
b) at least one monosaccharide residue comprising an amine group linked to a furanose or pyranose ring. The amine is a primary amine, glycosylamine or secondary amine, preferably a primary amine, preferably on a 2-position of furanose or pyranose ring.
   and first and second carbohydrate are covalently linked (directly or through a spacer) to each other,
   with the provision that at least one carbonyl group or amine group of one of the carbohydrates is linked to the carbonyl or amine group or a hydroxyl group of the other carbohydrate,
   and a carbonyl group being
1) an aldehyde or ketone is changed in the conjugation to a derivative of aldehyde or ketone including oximes, Schiff bases:
   and/or
2) a carboxylic acid is changed to an amide or an ester and/or an amine is derived to
3) a Schiff base; or an amine by reduction, or amide.

The invention is especially directed to conjugates, wherein first carbohydrate is biorecognition group and second carbohydrate is backbone PO as described in Formula I and other Formulas according to the invention.

The present invention is thus directed to synthesis of carbohydrate conjugates by conjugating the first carbohydrate with the second carbohydrate, preferably from the preferred reactive groups such as chemoselective ligation groups.

Linkages Only from Carbonyls or Amines of Carbohydrate 1 and 2

In a preferred embodiment at least one carbonyl group or amine group of the first carbohydrate is linked to the carbonyl or amine group of the second carbohydrate. The preferred subgroups of these structures includes linkages comprising a spacer and linkages directly from one functional group to another functional group. In a preferred embodiment carbonyl group of one carbohydrate is linked to an amine group of another carbohydrate.

In a preferred embodiment an uronic acid group of one carbohydrate is linked to an amine on second carbohydrate, preferably to a secondary amine, more preferably amine on 2-position of a residue (such as amine of glucosamine GlcN or galactosamine GalN), or to glycosylamine at the reducing end of the other carbohydrate.

More preferably a glycosaminiglycan carbohydrate is modified by at least, one more preferably by at least two glycosylamines and even more preferably by at least three and even more preferably by at least four glycosylamines and in a separate embodiment preferred oligovalent oligomers from 2-10, preferably 3-10, or 3-8, more preferably 4-10 or 4-8 glycosylamines or polymer comprising more than 10 glycosylamines, but in a preferred embodiment less than 10 million and in seperate embodiment 10 to million or 10 to 100 000, and in another preferred embodiment 10-1000 or even more preferably smaller polyvalen conjugates comprising 10-100 glycosylamines. The preferred glycosylamines linked to the glycosamino glycan include monosaccharides (preferably in oligovalent or polyvalent form) and oligosaccharide comprising 2-10 monosaccharide residues (preferably in oligovalent or polyvalent form).

### Carbohydrates Comprising 6-Modification, Preferably a Carbonyl or Ester Modifications

The invention is especially directed to novel biorecognition conjugates produced by modification of 6-position of a pyranose formed monosaccharide residue, preferably a hexose or hexosamine or derivative thereof, especially when 6-position comprises a carbonyl structure (a double bonded oxygen linked to the carbon atom), the carbonyl structure preferably being an aldehyde, ketone or the carbonyl being carboxylic acid structure of an uronic acid structure.

### Preferred Uronic Acid Comprising Structures

In a preferred embodiment glucopyranose or galactopyranose comprises the carbonyl structure. The preferred residues are uronic acid GlcA, GlcANAc (uronic acid derivative of GlcNAc), GalA, and GalANAc (uronic acid derivative of GalNAc).

### Preferred Conjugates

Conjugates from Uronic Acids and Production Thereof.

The invention revealed novel uronic acid based glycan structures, wherein the carboxylic acid group of the uronic acid is conjugated directly or by a spacer to another carbohydrate. The preferred linkages to the uronic acid structure are ester and amide linkages to a spacer or hydroxyl or amino group of a carbohydrate.

The invention is further directed to specific oxidation methods to produce carboxylic acid from 6-hydroxyl groups of various carbohydrates, especially from cyclodextrins and/or glycosaminoglycans. The invention is further directed specific activation of carboxylic acid structures for conjugation of the glycans, the invention is especially directed to activation of carboxylic acids as uronium structures, especially preferred carboxylic acid activators include e.g. HBTU, ByBUP and DMT-MM, and equivalents thereof, especially HBTU type activator is preferred, especially with a second activating reagent such as tertiary amines include e.g. diisopropylethylamine and trimethylamine, especially DIPEA type reagents.

Conjugates from Uronic Acids of Cyclodextrins and Production Thereof.

The invention is directed to novel uronic acid comprising structures, wherein the carboxylic acid group of the uronic acid is conjugated directly or by a spacer to another carbohydrate. The preferred linkages to the uronic acid structure are ester and amide linkages to a spacer or hydroxyl or amino group of a carbohydrate.

### Preferred Carbohydrate Uronic Acid Ester Conjugates

The invention is in a preferred embodiment directed to carbohydrates, wherein a carboxylic acid from a spacer or another carbohydrate, preferably from spacer, is esterified to the 6-hydroxyl(s) of the carbohydrate, preferably as 6-hydroxyl of Glc residue(s) of oxidized glucose polymers or oligomers, such as alfa- or beta glucans or more preferably cyclodextrins or in glycosaminoglycans GlcN(Ac)₀ₒᵣ₁ residues of hyaluronic acid or heparin or keratan sulfate or poly-Nacetyllactosamines, or heparan sulfate or GalN(Ac)₀ₒᵣ₁ residues of chondroitin, chondroitin sulfates or dermantan sulfate.

### Preferred Cyclodextrin Uronic Acid Ester Conjugates

The invention is in a preferred embodiment directed to cyclodectrins, wherein a carboxylic acid from a spacer or another carbohydrate, preferably from spacer, is esterified to the 6-hydroxyl(s) of cyclodextrin.

### Preferred Esterification Conditions

Preferred esters in conjugates according to the invention are performed with reagents activating the esterification reactions, preferred activating reagents includes HBTU and diisopropylamine-type or analogous carboxylic acid activating regents preferably HBTU and diisopropylamine. Preferred solvent for conjugation is a dry solvent (containing no or very amount amounts of water and/or containing a water absorbing reagent suitable for the reaction) preferably the solvent is apolar solvent analogous to dry pyridine, more preferably the solvent is dry pyridine. Other type of preferred esterification reagents includes anhydrides of carboxylic acid such as divalent carboxylic acid, most preferably succininc acid anhydride.

### Preferred Carbohydrate Uronic Acid Amide Conjugates

The invention is especially directed carbohydrate carboxylic acid derivatives; especially glycosaminoglycan (hyaluronic acid, chodroitin (non-sulfated), chondroitin sulfates, heparan sulfates) or cyclodextrin derivatives; conjugated from the carboxylic acid residue to an amine in a spacer which is further conjugated

a) from an amine in the spacer to a reducing end carbonyl aldehyde of carbohydrate bioactive group, e.g by reductive amination, or by amidation to reducing end carboxylic acid (onic acid obtainable e.g. by oxidation of reducing end aldehyde to carboxylic acid by halogen such as iodide)
b) from a carboxylic acid or aldehyde or ketone in the spacer to reducing end glycosylamine of the second carbohydrate/a carbodydrate bioactive group.
c) from a carboxylic acid or aldehyde or ketone in the spacer to secondary or primary amine of a monosaccharide reside preferably to 2-position amine of hexosamine such as GlcN or GalN, of the second carbohydrate/a carbodydrate bioactive group.
Preferred Conjugates from Secondary Amine of Carbohydrate

The invention is further directed to conjugates from secondary amines of carbohydrates. The preferred conjugates includes conjugates of amines of chitosan or glyccosaminoglycans. It is realized that glycosaminoglycans are especially preferred because of low immunogenicity for in vivo applications. The chitosan are preferred for immunostimulation application especially for vaccines. Among the glycos aminoglycan especially preferred are conjugates hyaluronic acid, chondroitin (/sulfate), and dermantan are preferred in a specific embodiment, especially chondroitin (/sulfate), and dermantan with novel deacetylated amine structures, in a preferred embodiment the glycosamino glycan comprises sulfates.

The conjugates comprise a linkage
a) from a carboxylic acid or aldehyde or ketone in the spacer to secondary or primary amine of a monosaccharide reside preferably to 2-position amine of hexosamine such as GlcN or GalN, of the second carbohydrate/a carbodydrate bioactive group and
b) a preferred linkage to amine or carbonyl or ester

### Preferred Spacers

In a preferred embodiment the spacer comprises a amino-oxy or methylaminooxygroup reactive with an aldehyde or ketone of one carbohydrate in a preferred embodiment reducing end aldehyde or 6-position aldehyde or a ketone acid amidated to amine of hexosamine (GalN or GlcN) and a second reactive group reactive to carbonyl or amine in another carbohydrate. Other preferred spacers include spacers with one amine group and a carbonyl group or two amine groups or two carboxylic acid groups.

The invention is especially directed to chitosan or glycosaminoglycan amino-oxy conjugates when the spacer is conjugated secondary aminogroup of the carbohydrate and from another end of the spacer to the other carbohydrate preferably to 6-position carbonyl group of the second carbohydrate or secondary amine in the second carbohydrate.

### Preferred Cyclodextrins

The invention is especially directed to conjugates from 6-position hydroxyl ester or carbonyl (aldehyde or carboxylic acid) on 6-position of cyclodextrin.

### Preferred Chitosan Conjugates

The invention is especially directed to conjugates from secondary amines of chitosan to non-reducing end structures of second carbohydrate such as on 6-position carbonyl of second carbohydrate or secondary amine.

### Preferred Sized of Longer Backbone Polysaccharides

The invention is preferably directed to conjugates of glycosamino glycan with sizes allowing effective serum retention of a bioactive molecule(s) such an oligosaccharide (preferably in polyvalent form), or glycoprotein (preferably small Mw glycoprotein such as cytokines or growth factors). Preferred carbohydrate sizes for backbones includes oligosaccharides with 5-100 monosaccharide residues, more preferably 5 to 25 monosaccharide residues.

### Additional Molecular Components

In addition to the above-mentioned compounds, it is possible to couple substituents to the compound such as a marker group or label, and/or a drug or other active agent. Generally, the marker group or label makes it possible to use the carbohydrate-containing backbone carbohydrates for in vitro or in vivo diagnoses. The coupling of the marker to the backbone carbohydrates generally takes place via covalent bonds. Markers known to the skilled artisan for use in in vivo diagnosis may be employed for this purpose, such as, for example, radioactive markers which contain a bound radionuclide (e.g., technetium), X-ray contrast agents (e.g., iodinated compounds), as well as magnetic resonance contrast agents (e.g., gadolinium compounds). The relative proportion of marker to the entire molecule generally less than about 1% in terms of molecular weight.

In selecting a drug for coupling to the backbone carbohydrates moiety, the drug would be chosen in reference to the particular disorder to be treated and the regimen involved. The coupling of the drug to the backbone carbohydrates generally occurs through covalent or ionic bonds. Exemplary drugs which could be bound to the carbohydrate-containing backbone carbohydrates of this invention include:

antitumor agents such as, for example, daunomycin, doxorubicin, vinblastine, bleomycin;
antibiotics such as, for example, penicillins, erythromycins, azidamfenicol, cefalotin and griseofulvin;
immunomodulators such as, for example, FK-506, azathioprine, levamisole;
antagonists of blood platelet activation factors;
leukotriene antagonists;
inhibitors of the cyclooxygenase system such as, for example, salicylic acid compounds;
lipoxygenase inhibitors,
antiinflammatory agents such as, for example, indomethacin;
antirheumatic agents such as, for example, nifenazone.

Advantageously, the carbohydrate-containing backbone carbohydrates of this invention are able to react with all naturally occurring receptors which specifically recognize in vivo the biorecognition molecule of ligands. These preferably are receptors which are expressed on cell surfaces, for example, by mammalian cells including human cells, bacterial cells or viruses. Also preferred are hormones and toxins, and recognizing receptors which recognize hormones or toxins. Particularly preferred cell surface receptors are those which belong to the class of selectins. Most particularly preferred receptors are those expressed in inflammatory disorders, for example Leu-8 (=L-selectin=gp90.sup.mel=LAM-1=LEC-CAM-1), ELAM-1 (=E-selectin) and GMP-140 (=P-selectin=CD62=PADFEM).

If the carbohydrate-containing backbone carbohydrates of this invention are employed as antiadhesion therapeutic agents, the aim is that, in the case of inflammations, they prevent the ELAM-1 receptors on stimulated the surface of leukocytes. In the case of influenza therapy, the carbohydrate-containing molecules prevent the adhesion of viruses to the neuraminic acid on the cell surface and thus also the endocytosis of the virus particles.

### Preparation of the Conjugates

The present invention also relates to a process for the preparation of the conjugates as defined above, comprising
a) reacting an oligosaccharide PO carrying a spacer group (y′)ₚ-(S)_{q}-(z)ᵣ- with a compound of the formula Bio-Y″ wherein PO, Bio, S, p, q, z, and r have the meanings given above, Y″ is a reactive group, such as an amino, hydroxy, carboxylic acid, activated ester, aldehyde or a keto group, and y′ is a group capable of reacting with the group Y″ on the Bio group to form the linkage y, wherein y means the same as above, or
b) reacting a compound having the formula Bio-(y)ₚ-(S)_{q}-(z′)ᵣ with an oligosaccharide PO having a reactive group X″, such as an amino, hydroxy, carboxylic acid, activated ester, aldehyde or a keto group, wherein PO, Bio, S, p, q, y, r and n have the meanings given above, and z′ is a group capable of reacting with X″ to form the linkage z, wherein z means the same as above.

Specifically, the present invention also relates to a method for the preparation of the polyvalent constructs comprising reacting a carrier structure carrying a reactive group X″, such as a hydroxy, amino, carboxylic acid, activated ester, aldehyde or a keto group, in a first step with a spacer forming compound of the formula (y′)ₚ-(S)_{q}-(z′)ᵣ wherein S, p, q and r have the meanings given above and z′ is a group capable of reacting with X″ to form the linkage z and y′ is a group capable of reacting with a reactive group on the Bio group to form the linkage y, which PO spacer construct obtained is thereafter reacted with a compound Bio-Y″ wherein Y″ is a reactive group as defined above for X″, to form the conjugate according to the invention.

It is also possible to react in a first step, the bioreactive compound Bio-Y″ with the desired spacer (y′)ₚ-(S)_{q}-(z′)ᵣ, which Bio-spacer construct then in a second step, is reacted with the desired oligomer PO carrying a reactive group to form the end conjugate.

According to a preferred embodiment of the invention the polyvalent structure is made by first synthesizing the linking structure on the carrier structure PO. When PO is a polysaccharide structure, such as a chitosan structure, it can be reacted with the spacer structure in unprotected form, for example an amino group on the polysaccharide can be reacted with a spacer containing a carboxylic acid group to form an amide bond. Thereafter the conjugate containing the polysaccharide with attached spacer groups carrying an O-substituted hydroxylamine, i.e. an amino-oxy group at the end of the spacer is reacted with a desired biorecognition group, for example an oligosaccharide, containing e.g. an aldehyde or keto group, at the reducing end thereof, to form the desired O-hydroxylamine linkage. This reaction is preferably carried out in a buffered aqueous solution, without additional solvents. In some cases the reactions may need to be performed in the presence of a small amount, such as at the most appr. 10%, of a polar solvent, which would not precipitate the reagents and react with O-hydroxylamine group. Such solvents include acetonitrile. The solvent may be needed for facilitation of the solubilization of the Bio-group if it would not be easily water soluble.

The coupling of carbohydrate substances according to the present invention to polyvalent carriers has several benefits. Firstly it allows conserving the reducing end ring structure of the carbohydrate substance at least partially. For monosaccharide and oligosaccharide substances this could be important for preserving the biological activity of the substances. For polysaccharide structures, preserving the reducing end structure limits the spacing between the polysaccharide substances and prevents crowding from the polymer backbone, which could prevent the most effective activity or bioactivity close to the polymer.

According to the present invention the conjugation reactions can be performed in polar solvent systems in which both the backbone carbohydrate and the carbohydrate to be conjugated are soluble. Preferably the reactions are performed in aqueous solutions comprising less than 50% of organic solvent, more preferably less than 30% of organic solvent and even more preferably less than 10% of organic solvent. The organic solvent, if used or present, is preferably a non-reactive solvent, The possible organic solvents are used in amounts that will not precipitate the carbohydrate reagents. Most preferably the present invention uses an aqueous solution comprising no organic solvents or only negligible amounts of organic solvents.

Preferably the conjugation reactions are performed in a buffered solvent system. Such reactions are preferably performed at pH-values between 3-7, more preferably in the pH range of 3.5-6.5, and even more preferably at a pH about 3.8-5.5 and most preferably within pH range of 3.8-4.5. In a preferred embodiment the reactions are preformed at about pH 4. Preferably the reactions are performed at pH 4 in an aqueous buffer comprising no organic solvent, and preferably a carboxylic acid buffer is used, such carboxylic acid buffer being e.g. an acetate buffer. In a preferred embodiment, the acetate buffer has a concentration within the range of 0.10-0.30 M, more preferably the acetate buffer is about 0.2 M acetate buffer.

Carbohydrate nomenclature is essentially according to recommendations by the IU-PAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that the monosaccharide residues on a oligo- or polysaccharide chain are preferably in the forms these occur in natural polysaccharides, preferably in forms of human and/or animal polysaccharides if the polysaccharide occur in human or animal, Gal, GalN, GalNAc, Gal A, Glc, GlcN, GlcNAc, GlcA, Man and sialic acids such as Neu5Ac, and NeuGc are preferably of the D-configuration, Fuc of the L-configuration, and all the glycosidic monosaccharide units are preferably in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues α3 and α6 mean the same as α2-3 and α2-6, respectively, and with other monosaccharide residues α1-3, β1-3, β1-4, α1-4 and β1-6 can be shortened as α3, β3, α4, β4, and β6, respectively. Lactosamine refers to N-acetyllactosamine, Galβ4GlcNAc, and sialic acid is N-acetylneuraminic acid (Neu5Ac) or N-glycolylneuraminic acid (Neu5Gc) or any other natural sialic acid.

Processes for the chemical, enzymatic or chemoenzymatic synthesis of carbohydrates which are recognized by cell surface receptors are known to the skilled worker from the chemical literature and from review articles. For the chemical synthesis for example CARBOHYDRATE RESEARCH, Elsevier Science Publishers V. U., Amsterdam, Journal of Carbohydrate Chemistry, Marcel Dekker, Inc., New York; H. Paulsen, Angewandte Chemie 94:184 (1982); R. R. Schmidt, Angewandte Chemie, 98:213 (1987); H. Kunz, Angewandte Chemie 44:247 (1987); H. Paulsen, Angewandte Chemie 102:851 (1990). For the enzymatic synthesis for example CARBOHYDRATE RESEARCH, Elsevier Science Publishers B. U., Amsterdam; Journal of Carbohydrate Chemistry, Marcel Dekker, Inc., New York; Carbohydrate Chemistry, Marcel Dekker, Inc., New York; David et al., Advances in Carbohydrate Chemistry and Biochemistry 49:1975 (1991); Nielsson, Applied Biocatalysis 1:117 (1991); Drueckhammer et al., Synthesis 499 (1991). Chemoenzymatic synthesis is defined as the combination of chemical and enzymatic reaction steps for the synthesis of the biorecognition molecule and of a covalent linkage of biorecognition molecule and spacer.

The coupling of the biorecognition molecule with a free reducing end to the spacer takes place via a covalent bond. The following processes can be carried out for the coupling:

The oligosaccharide with free reducing end is converted, for example, by analogy to the process of Lundquist et al., J. Carbohydrate Chem. 10:377 (1991), into the free 1-amino-glycoside which is subsequently covalently linked to a spacer by acylation. Alternatively, the compound may be covalently linked, for example, by analogy to the process of Kochetkow, Carbohydrate Research 146:C1 (1986), to the spacer using an N-hydroxysuccinimide active ester as activated group on the spacer.

The free reducing end of the oligosaccharide can be converted to a lactone, by analogy, to the process of Isebell et al., in METHODS OF CARBOHYDRATE CHEMISTRY, Academic Press, New York (1962), using iodine and potassium hydroxide. This lactone can be covalently linked to the spacer, for example, by means of a primary amino group which is a component of the latter. Id.

The formation of a covalent bond between the reducing end of an oligosaccharide and the spacer also is possible by reductive amination. This method employs a spacer having a primary amino group at the appropriate end, for example, by analogy to Lane, Synthesis 135 (1975).

If the oligosaccharide contains, at its reducing end, an amino sugar with a free amino group, the latter can be covalently linked to the spacer, for example, by analogy to the process of Kochetkow, Carbohydrate Research 146:C1 (1986), by means of an N-hydroxysuccinimide active ester of the latter.

### Pharmaceuticals

The pharmaceutical products are preferably produced and administered in dosage units. Preferred in the case of solid dosage units are tablets, capsules and suppositories. Examples of suitable solid or liquid pharmaceutical presentations are granules, powders, tablets, coated tablets, (micro)capsules, suppositories, syrups, emulsions, suspensions, aerosols, drops or injectable solutions in ampoule form as well as products with protracted release of active substance, in the production of which it is customary to use excipients and additives and/or aids such as disintegrants, binders, coating agents, swelling agents, glidants or lubricants, flavorings, thickeners or solubilizers. Examples of commonly used excipients or ancillary substances which may be included are magnesium carbonate, titanium dioxide, lactose, mannitol and other sugars, talc, lactalbumin, gelatin, starch, vitamins, cellulose and its derivatives, animal and vegetable oils, polyethylene glycols and solvents such as, for example, sterile water, alcohols, glycerol and polyhydric alcohols.

The pharmaceuticals according to the invention are generally administered intravenously, orally or parenterally or as implants, but rectal use is also feasible. The daily doses necessary for the treatment of a patient vary depending on the activity of the molecule, the mode of administration, the nature and severity of the disorder and the age and body weight of the patient, etc. The daily dose can be administered either by a single administration, in the form of a single dosage unit, or as a plurality of small dosage units or by multiple administration of divided doses at particular intervals. The daily dose change during the course of treatment depending on the number of receptors expressed during a particular phase of the disease. It is conceivable that only a few receptors are expressed on the cell surface in the initial stage of a disease and, accordingly, the daily dose to be administered would be less than that for patients suffering a well-progressed disease.

The following examples illustrate the invention without limiting the same in any way.

### EXAMPLES

In the examples the following carbohydrates and methods were used:

Carbohydrates: Lewis b hexasaccharide LNDFH I (Fucα1-2Galβ1-3(Fucα1-4)GlcNAcβ1-3Galβ1-4Glc) was purchased from IsoSep (Lund, Sweden). LNnT (Galβ1-4GlcNAcβ1-3Galβ1-4Glc) and GnLacNAcLac (GlcNAcβ1-3Galβ1-4GlcNAcβ1-3Galβ1-4Glc) were from Kyowa Hakko (Japan). Chondroitin sulphate A (CSA) (bovine trachea), γ-CD, and para-nitrophenyl-β-glucuronide (pNP-β-GlcA were from Calbiochem.}

Chromatographic Methods. Gel filtration chromatography was performed with Superdex™ Peptide HR 10/30 (10×300 mm) (Amersham Pharmacia Biotech, Sweden) with 200 mM NH₄HCO₃ as eluent, at a flow rate of 1 ml/min or Superdex 30 5/95 (5×95 cm) with 200 mM NH₄HCO₃ as eluent, at a flow rate of 5 ml/min. All experiments were monitored at 214 nm. Fractions of 10 ml were collected in Superdex 30 runs.

Nuclear magnetic resonance spectroscopy. Prior to one dimensional ¹H NMR experiments, the samples were lyophilized twice from D₂O (99.9%) (Aldrich) and then dissolved in 38 μl D₂O. The ¹H NMR spectra were recorded with a Varian Unity 500 spectrometer (Varian Inc. CA, USA) at 23° C. using a gHX nano-NMR probe (Varian Inc. CA, USA). The ¹H chemical shifts are presented by reference to internal acetone (δ=2.225 ppm).

Mass spectroscopy. Matrix-assisted laser desorption/ionization time-of-flight (MALDI-TOF) mass spectra (MS) were recorded on a Voyager-DE™ STR Bio-Spectrometry™ (PerSeptive Biosystems) time-of-flight instrument. Samples were analyzed in either positive ion delayed extraction reflector mode using 2,5-dihydroxybenzoic acid (DHB) (Aldrich) matrix (10 mg/ml in H₂O) or negative ion delayed extraction linear mode using 2,4,6-trihydroxyacetophenone (THAP) (Fluka) (3 mg/ml in acetonitrile/20 mM aqueous diammonium citrate, 1:1, by volume).

### Example 1

### Chondroitin Sulphate

### 2.2. Desulphation and Acid Hydrolysis of Chondroitin Sulphate

Desulphation of chondroitin sulphate A (CSA) was carried out essentially as described previously (Nagasawa, Inoue & Tokuyasu, 1979). Pyridinium salt of CSA was prepared by passing the sample in water through a cation exchange column (AG50W-X8, 200-400 mesh, hydrogen form) (Bio-Rad) at room temperature. The eluate was neutralized with pyridine and dried by rotary evaporator. The obtained pyridinium salt of CSA was dissolved in DMSO containing 10% of methanol and incubated for 5 hours at 80° C. Reaction was terminated by cooling and the content was diluted with water to dimethyl sulphoxide (DMSO) concentration <5% (v/v). The solution was then adjusted to pH 9.0-9.5 with NaOH and dialyzed in a regenerated cellulose tubing (MWCO 6000-8000) against running tap-water for 5 hours and then against distilled water overnight. The dialyzed desulphated CS was dried by rotary evaporator.

Desulphated CS was partially hydrolyzed in 0.5 M TFA for 20 h at 60° C. Reaction was terminated by cooling, concentrated to 20 ml and then adjusted to pH 8 with 1 M NH₄HCO₃. Hydrolyzed chondroitin was fractionated with a column of Superdex 30 (5×95 cm) eluted with 200 mM NH₄HCO₃ and the eluate was monitored at 214 nm. Fractions were analyzed by mass spectrometry. Quantification was performed by UV-absorbance comparison to external glucuronic acid and N-acetylglucosamine standards.

### 2.4. Formation of LNnT-Glycosylamine

LNnT was converted to glycosylamine form (LNnT-NH₂) essentially as described previously (Tamura et al., 1994) by incubating LNnT in 1 μmol aliquots in saturated NH₄HCO₃ and incubating samples at 50° C. for 24 h. LNnT-NH₂ was recovered by repeated lyophilization from 10 μl H₂O until no NH₄HCO₃ was visualized.

### 2.5. Amidation of LNnT-Glycosylamine to Chondroitin Oligomer

Desulfated chondroitin 14-mer (Ch14) was amidated using LNnT-NH₂ as follows: Chondroitin 14-mer (150 nmol), LNnT-NH₂ (10 μmol), HBTU (10 μmol) (Novabiochem), and DIPEA (N-ethyldiisopropylamine) (10 μmol) (Fluka Chemika) were dissolved in dry pyridine (2.35 ml). Reaction was performed at room temperature, in the dark and in constant magnetic stirring for four days. Reaction mixture was then dried in a rotary evaporator, followed by addition of 5 ml of methanol and evaporation repeated three times. Sample was purified in three experiments using Superdex Peptide, and fraction contents were verified using MALDI, then pooled.

### Example 2

### γ-CD

### 2.3. Oxidation

Selective oxidation of primary alcohol groups of γ-CD with TEMPO (2,2,6,6-tetramethylpiperidine-1-oxy radical) (Aldrich) catalysis was carried out essentially as described previously (Fraschini & Vignon, 2000). Briefly, 300 μmol of γ-CD, 62.4 μmol of TEMPO, and 1920 μmol of NaBr were dissolved in 90 ml of 0.2 M Na-carbonate buffer, pH 10. The solution was cooled on ice and 3.36 mmol of Na—ClO was added in several portions. The reaction was allowed to proceed for 10 min on ice. Remaining aldehyde groups were reduced by adding 2.4 mmol NaBH₄ and incubating for 1 hour. The sample was neutralized to pH 7 with 4 M HCl. The oxidized γ-CD species (ox-γ-CD) were isolated by gel filtration chromatography on a column of Superdex 30 (5×95 cm) eluted with 200 mM NH₄HCO₃. The eluent was monitored at 214 nm and selected fractions were analyzed by mass spectrometry. Quantification of products was performed by UV-absorbance comparison to external glucuronic acid standard.

### 2.4. Formation of LNnT-Glycosylamine

LNnT was converted to glycosylamine form (LNnT-NH₂) essentially as described previously (Tamura et al., 1994) by incubating LNnT in 1 μmol aliquots in saturated NH₄HCO₃ and incubating samples at 50° C. for 24 h. LNnT-NH₂ was recovered by repeated lyophilization from 10 μl H₂O until no NH₄HCO₃ was visualized.

### 2.5. Amidation of LNnT-Glycosylamine to Chondroitin Oligomer and ox-γ-CD

The oxidized γ-CD was amidated using LNnT-NH₂ as follows: ox-γ-CD (200 nmol), LNnT-NH₂ (10 μmol), HBTU (10 μmol), and DIPEA (10 μmol) were dissolved in dry pyridine (3 ml). Reaction was performed, purified and verified as described above for Ch14.

### 2.6. Esterification

Boc-aminooxyacetic acid (Boc-Aoa) (Novabiochem) was ester-linked to γ-cyclodextrin (γ-CD) by dissolving 20 μmol γ-CD, 1.6 mmol Boc-Aoa, 1.6 mmol HBTU, and 1.6 mmol DIPEA in pyridine (40 ml). Reaction was performed at room temperature, in the dark, and in constant magnetic stirring for two days. Reaction mixture was then dried in a rotary evaporator, followed by addition of 10 ml of methanol and evaporation repeated three times. Sample was dissolved in 10% ethanol and centrifuged 4000 rpm for 10 min at room temperature. Supernatant was transferred into a cellophane tube (MWCO 500), dialyzed against running tap-water for 4 hours, then against 20% ethanol/10 mM NH₄Ac pH 5 for two days with one change of solution, and finally dried by a rotary evaporator.

Boc was removed from aliquots of sample just prior to oxime reaction by dissolving 10 μmol Boc-Aoa-γ-CD in 10 ml of TFA (Aldrich) and incubating for 10 min at room temperature. Solution was dried by a rotary evaporator, followed by addition of 10 ml of methanol and evaporation repeated three times.

### 2.7. Oxime Formation

LNnT was linked to γ-CD in an ester linked oxime-bridge. 10 μmol Aoa-γ-CD and 1630 μmol LNnT (Kyowa Hakko, Japan) were dissolved in 12 ml 0.2 M Na-acetate pH 4 and pH was adjusted to pH 4 by adding 700 μl 0.5 M Na-acetate pH 5.5. The reaction was allowed to proceed at room temperature, under constant magnetic stirring for 15 hours. The reaction mixture was fractionated in three runs using Superdex 30 (5×95 cm, Amersham Pharmacia Biotech, Sweden) in 10 mM NH₄Ac, pH 5.0.

### Example 3

### 2.8. Stability Analysis of the Oxime-Linkage

To analyze the stability of oxime-bond we generated a mixture of LNnT modified using aminooxyacetic acid (LNnT-Aoa) and LNnT: 50 μmol of LNnT and 100 μmol Aoa (Sigma) were dissolved in 1.2 ml of 0.2 M Na-acetate buffer, pH 4.0 and the reaction was allowed to proceed at room temperature for 48 hours. This reaction resulted in a mixture containing LNnT-Aoa and LNnT in a molar ratio of 60/40. The sample was desalted using gel filtration chromatography and aliquots of 100 nmol were incubated in 1.0 M and 0.1 M HCl (pH 0 or pH 1, respectively) at room temperature and at +37° C. Aliquots were removed at selected time points and analyzed by MALDI-TOF MS.

### Results and Discussion to Examples 1-3

### Generation of Chondroitin Oligomer

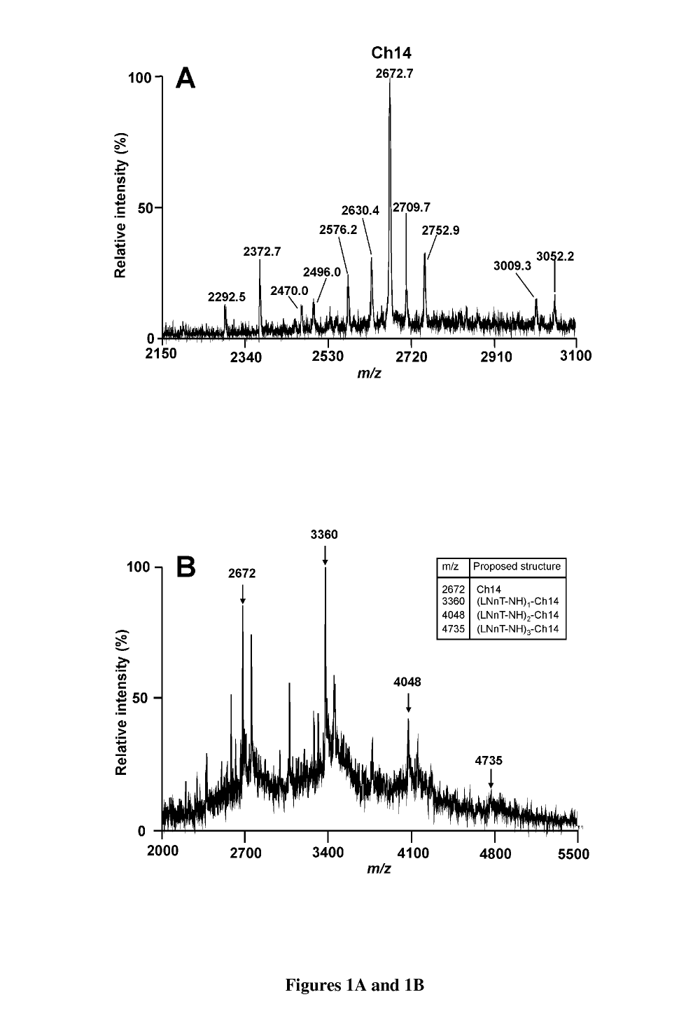

To construct a linear multivalent molecule, chondroitin sulphate A oligomer was prepared to act as a carrier. Acid hydrolysate of desulphated chondroitin sulphate A (from bovine trachea) (Scheme 1) (Examples 1-3) was fractionated by gel filtration. Mass spectrometry was used to verify fraction peak contents and fractions containing 10-16-mers were pooled and re-fractionated as above. Fractions containing chondroitin 14-mer (Ch14, compound 2) as the major compound were pooled and analyzed using MALDI-TOF MS in the linear negative mode (FIG. 1A). The signals were identified as chondroitin 12-mer (m/z 2292.5 [M-H]⁻, 2372.7 [M-H+SO₃]⁻), chondroitin 14-mer (m/z 2672.1 [M-H]⁻, 2752.9 [M-H+SO₃]⁻, 2630.4 [M-H−Ac]⁻, 2709.7 [M-H+SO₃−Ac]⁻), and chondroitin 16-mer (m/z 3052.2 [M-H]⁻, 3009.3 [M-H−Ac]⁻. In addition, minor signals representing chondroitin 13-mer (GalNAc₇GlcA₆) m/z 2496.0 [M-H]⁻, and 2576.2 [M-H+SO₃]⁻ were observed.

### Conjugation of LNnT-NH₂ to Chondroitin Oligomer

Amine function was introduced to LNnT by converting reducing end of the oligosaccharide to the glycosylamine. Resulting LNnT-NH₂ was then conjugated to Ch14 by amidation to GlcA carboxyl-groups (Scheme 1) in a reaction containing DIPEA as a catalyst and HBTU to create an oxoammonium ion. Reaction mixture was purified and fractionated by gel filtration. Fraction contents were verified using MALDI-TOF MS and multivalent products were pooled. The multivalent product (LNnT-NH-Ch14, compound 3) was analyzed using MALDI-TOF MS in the linear negative ion mode (FIG. 1B). The indicated signals were identified as Ch14 (m/z 2672 [M-H]⁻), (LNnT-NH)ᵢ-Ch14 (m/z 3360 [M-H]⁻), (LNnT-NH)₂-Ch14 (m/z 4048 [M-H]⁻), (LNnT-NH)₃-Ch14 (m/z 4735 [M-H]⁻), all proposed structures. The heterogeneity in the conjugate signals is due to chondroitin backbones of different sizes.

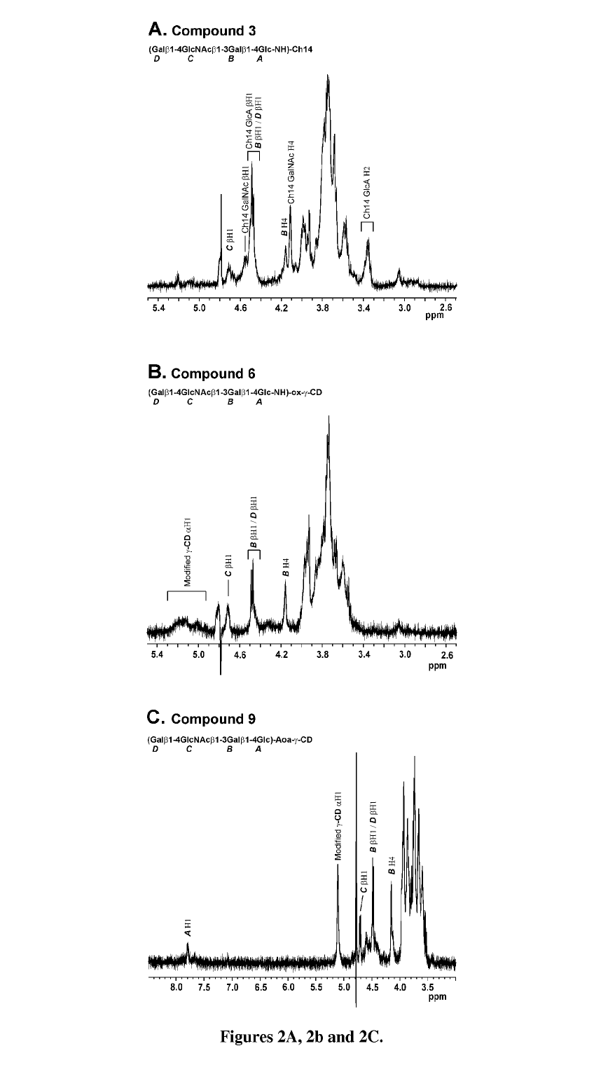

The ¹H NMR spectrum of LNnT-NH₂ linked to Ch14 backbone (LNnT-NH-Ch14, Compound 3) (FIG. 2A), show in the anomeric region H1 resonances βH1 of D-Gal and βH1 of C-GlcNAc, and H4 of 3-substituted B-Gal (4.159 ppm), consistent with those reported for the free LNnT molecule. Compared to the free LNnT the βH1 of B-Gal had shifted downfield, from 4.436 ppm to 4.49 ppm (overlapping with βH1 of D-Gal and βH1 of Ch14 GlcA) due to amidation of the A-Glc unit. All βH1 signals that originate from the chondroitin oligomer monosaccharide units resonate between 4.4-4.6 ppm. In addition, H4 signals of GalNAc and H2 signals of GlcA from the chondroitin oligomer are also seen. Importantly, the +/βH1 of A-Glc signals are missing indicating that no reducing LNnT is present in the sample. The average substitution level was 1.6 LNnT oligosaccharides per Ch14 molecule as calculated comparing the integrated intensities of LNnT N-acetyl proton signals and GlcA H2 signals of Ch14.

### Oxidation

Carboxylic acid groups were introduced to γ-CD by TEMPO catalyzed oxidation (Fraschini & Vignon, 2000) (Scheme 2). The conversion of alcohol groups to carboxylates proceeds via a reactive aldehyde-intermediates, which are present at low concentration throughout the oxidation. Consequently, the remaining aldehyde groups were reduced at the end of oxidation reaction using NaBH₄. A mixture of mono- to heptacarboxy-γ-CD was obtained and fractionated using gel filtration (data not shown). Fraction contents were verified using MALDI-TOF MS and fractions containing tetra- to heptacarboxy γ-CD were combined. The average oxidation level of γ-CD was 5 carboxylate groups as determined by MALDI-TOF MS analysis.

Conjugation of LNnT-NH₂ to ox-γ-CD

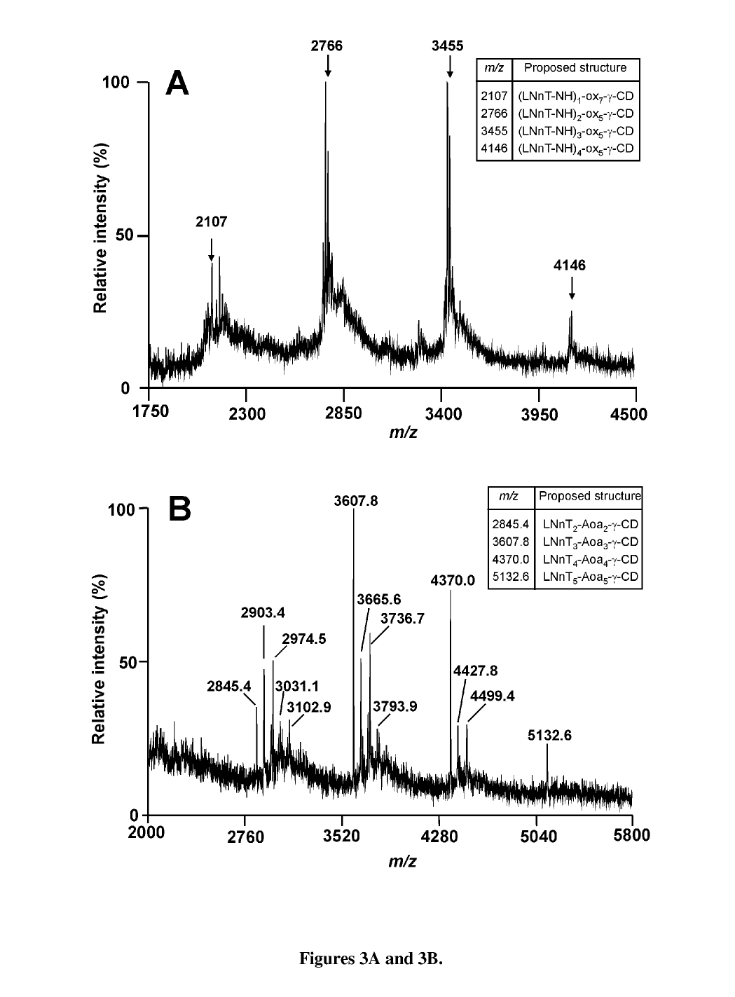

LNnT-NH₂ was conjugated to oxidized γ-CD (ox-γ-CD, compound 5) by amidation to 6′-position carboxyl-groups (Scheme 2) in a reaction containing DIPEA and HBTU. Reaction mixture was fractionated by gel filtration. Fraction contents were verified using MALDI-TOF MS and multivalent products were pooled. The multivalent product (LNnT-NH-ox-γ-CD, compound 6) was analyzed using MALDI-TOF MS in the reflector positive ion mode (FIG. 3A). The indicated signals were tentatively identified as (LNnT-NH)₁-ox₇-γ-CD (m/z 2107 [M+Na]⁺), (LNnT-NH)₂-ox₅-γ-CD (m/z 2766 [M+Na]⁺), (LNnT-NH)₃-ox₅-γ-CD (m/z 3455 [M+Na]⁺), (LNnT-NH)₄-ox₅-γ-CD (m/z 4146 [M+Na]⁺). The heterogeneity in the spectrum is due to variable levels of γ-CD oxidation.

The ¹H NMR spectrum of LNnT-NH₂ linked to oxidized γ-CD backbone (LNnT-NH-ox-γ-CD, Compound 6) (FIG. 2B), show in the anomeric region H1 resonances βH1 of D-Gal and βH1 of C-GlcNAc, and H4 of 3-substituted B-Gal (4.157 ppm) consistent with those reported for the free LNnT molecule. αH1 resonances of the modified γ-CD are seen around 5.126 ppm. When compared to the spectrum of unmodified γ-CD where αH1 signals (Glcα1-4) resonate at the same frequency (5.09 ppm), the αH-1 signal area of LNnT-NH-ox-γ-CD is very heterogenous due to the complex nature of the molecule. Compared to the free tetrasaccharide the βH1 of B-Gal had shifted downfield from 4.436 ppm to 4.48 ppm (overlapping with βH1 of D-Gal) due to amidation of the A-Glc unit as observed for LNnT-NH-Ch14. The α/βH-1 signals of A-Glc are missing indicating that no free reducing LNnT remains in the sample. The average substitution level could not be established from the spectrum because the heterogenous nature of the αH1 signals of the modified γ-CD resulted in unreliable integration of this area.

### Esterification and Oxime-Ligation

Boc-Aoa was ester-linked to 6′-position hydroxyl groups of γ-CD (Scheme 2) in dry pyridine. Reaction mixture was purified using dialysis. The average substitution level of Boc-Aoa was 3.5 as determined by MALDI-TOF MS analysis (data not shown).

To attach carbohydrate groups protecting Boc groups were removed from Boc-Aoa-γ-CD (Compound 7) using dry TFA after which chemoselective oxime ligation of LNnT to unprotected Aoa-γ-CD (Scheme 2, Compound 8) was performed in weakly acidic aqueous solution. The reaction mixture was fractionated using gel filtration chromatography and the multivalent product (Compound 9) was analyzed using MALDI-TOF-MS in the linear positive ion mode (FIG. 3B). The indicated signals were tentatively identified as LNnT₂-Aoa₂-γ-CD (m/z 2845.4 [M+Na]⁺), LNnT₃-Aoa₃-γ-CD (m/z 3607.8 [M+Na]⁺), LNnT₄-Aoa₄-γ-CD (m/z 4370.0 [M+Na]⁺), and LNnT₅-Aoa₅-γ-CD (m/z 5132.6 [M+Na]⁺). The heterogeneity in the conjugate signals is due to variable level of aminooxyacetic acid modification in LNnT-Aoa-γ-CD. In addition, molecular species were observed where the amine groups have probably been lost from the aminooxy units revealing hydroxyl groups (O—NH₂ converted to OH=m/z−15).

The ¹H NMR spectrum of LNnT linked to Aoa-γ-CD backbone (LNnT-Aoa-γ-CD, Compound 9) (FIG. 2C), show in the anomeric region H-1 resonances βH1 of D-Gal and βH1 of C-GlcNAc, and H4 of 3-substituted B-Gal at (4.152 ppm) consistent with those reported for the free LNnT molecule. αH1 resonances of the modified γ-CD are seen at 5.109 ppm. The βH1 signal for B-Gal when compared to free LNnT had shifted downfield from 4.436 ppm to 4.48 ppm due to the modification of A-Glc unit. Signal representing oxime proton A-Glc H1 is seen at 7.796 ppm. The α/βH-1 signals of A-Glc are missing indicating that no free reducing LNnT remains in the sample. The average substitution level was 3.1 LNnT oligosaccharides per modified γ-CD molecule as calculated by comparing the integrated intensities of LNnT βH1 of C-GlcNAc and αH1 signals of the modified γ-CD.

### Stability Analysis of the Oxime-Linkage Under Highly Acidic Conditions

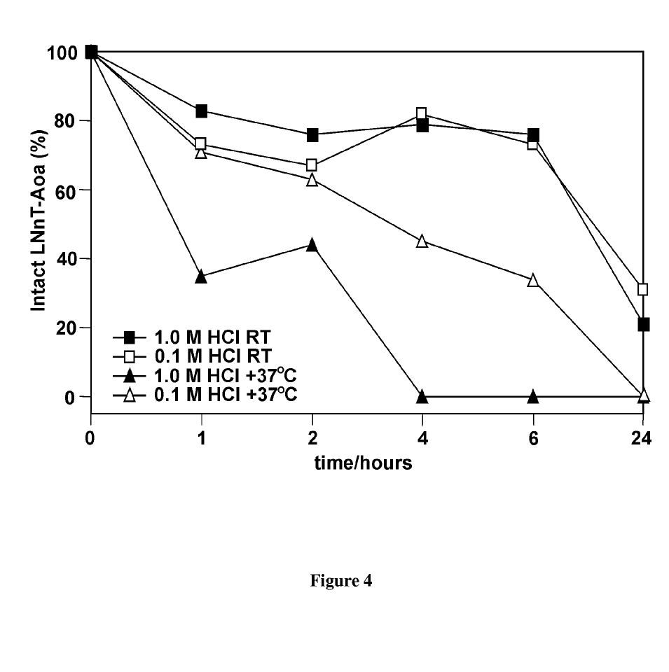

Stability of sugar oxime conjugates under highly acidic conditions was studied. Samples containing approximately 40% LNnT and 60% LNnT-Aoa were incubated in 1.0 M or 0.1 M HCl (at pH 0 or pH 1, respectively) at room temperature or at +37° C. At selected time points aliquots were removed and analyzed using MALDI-TOF MS. The relative amounts of LNnT and LNnT-Aoa were deduced from spectra (FIG. 4). Typically, orally administered molecules probably experience conditions in the stomach similar to +37° C. pH ˜1 studied here. At these conditions we found the half-life of approximately 3 hours for LNnT-Aoa. Even at +37° C. pH 0 half-life of about 1 hour was observed.

### 4. Discussion

The majority of infectious diseases are initiated by adhesion of pathogenic organisms to cells and tissues of the host (Ofek & Doyle, 1994). This adhesion is often mediated by lectins that bind to their complementary carbohydrate epitopes of glycoproteins or glycolipids on the surface of the host tissue. Therefore, prevention of adhesion or attachment of bacteria, viruses, or fungi to their host tissues, or detaching them from the tissue at the early stages of infection using carbohydrate based anti-adhesion therapy presents a highly promising approach. In addition, the alarming increase in antibiotic resistant bacterial pathogens makes it even more necessary to intensify the search for new means of combating bacteria.

In general, to design effective multivalent ligands, detailed information about the native structure of the binding proteins is needed. If this information is not available a number of scaffold systems presenting the carbohydrate ligand must be prepared. In addition, manipulation of the linker chemistry used to attach the carbohydrate epitope to its scaffold can have significant impact on the multivalent ligand affinity. We have in our studies focused on carbohydrate based architectures, where the conformational, constitutional, and configurational diversity of carbohydrates is used to control the presentation of carbohydrate ligands.

The present study presents conjugation of human milk tetrasaccharide LNnT through a β-glycosylamide linkage to scaffold molecules containing 6′-position carboxyl-groups. Two different scaffold molecules were used: A chondroitin 14-mer fraction and γ-cyclodextrin oxidized to express carboxyl groups. The chondroitin 14-mer fraction was isolated from an acid hydrolysate of desulphated chondroitin sulphate by gel filtration chromatography. Carboxyl groups were introduced to γ-cyclodextrin by oxidizing the primary hydroxyl groups by TEMPO oxidation (Fraschini & Vignon, 2000). In addition, we describe here a novel type of oxime-linked sugar-sugar conjugate that has not, to our knowledge, been previously described.

GAGs are excellent scaffold candidates for creating multivalent glycoconjugates because their carboxyl-groups can be either directly substituted by sugar moieties or functionalized for subsequent attachment of carbohydrate units. However, few studies showing GAG based oligosaccharide conjugates have been published. These include conversion of hyaluronan to its β-cyclodextrin derivate (Solies, 1999) and sialyl-Lewis x-heparin conjugates (Sakagami et al., 2000). In the present study a chondroitin 14-mer fraction was prepared, and subsequently substituted by the human milk tetrasaccharide LNnT. The tetrasaccharide was first converted to a glycosylamine form by incubation in saturated ammonium bicarbonate (Manger, Rademacher & Dwek, 1992), and the crude glycosylamine was amidated with the chondroitin oligomer carboxyl groups. Oligosaccharide derivatization through a β-glycosylamide linkage is an established method in glycobiology (Chiu, Thomas, Stubbs & Rice, 1995; Wong, Manger, Guile, Rademacher & Dwek, 1993). The present conjugates are to our knowledge the first where oligosaccharide glycosylamines have been conjugated directly to glycosaminoglycan chains by amidation, without including spacers. These conjugates have the advantage that their degradation products are devoid of any additional linker structures.

The amidation of oligosaccharide glycosylamine to the chondroitin oligomer proceeds with the current reaction conditions relatively slowly. The main product carried one oligosaccharide chain; di- and trisubstituted products were also present (about 15% and 5%, respectively, as deduced from the mass spectrum). Oxidized γ-cyclodextrin expressing 6′-carboxyl groups was however amidated more efficiently, yielding as the major products di- and trisubstituted species. With a similar methodology, synthesis of glycosylated calixarene through the formation of amide bonds using calix[4]arene diacid and galactosamine has been attempted. It is noteworthy that in this study steric effects prevented the coupling when using simple aminoglycosides and longer spacers were needed for successful reactions (Schadel, Sansone, Casnati & Ungaro, 2005). Other carboxyl activators may yield even higher derivatization levels with the present scaffold types. Carbodiimides are reportedly poor activators in uronic acid amidations (Pumphrey, Theus, Li, Parrish & Sanderson, 2002), but the activity of e.g. DMTMM (Sekiya, Wada & Tanaka., 2005) and HBPyU (Baisch & Ohrlein, 1998) remains to be established.

The chondroitin oligomer based conjugates present their oligosaccharide ligands on a linear scaffold, which may mimic e.g. natural mucins and polylactosaminoglycans.

These may find preferential use in e.g. selectin inhibitor area: Polyvalent sialyl-Lewis x conjugates based on polylactosamine (Renkonen et al., 1997) or mucin type (Satomaa, 2000) scaffolds have been shown to bind selectins with high affinity. The method described here can also be used to create multivalent molecules on other GAG structures. Although GAG materials can be obtained in good quantities from animal sources, biotechnologically produced GAGs would be preferred. Indeed, GAG type polysaccharides are available biotechnologically from E. coli K4 (Volpi, 2003) and K5 (Lindahl et al., 2005) capsular polysaccharides.

Synthesis of several neoglycoconjugates based on cyclodextrin scaffold having one or many carbohydrates attached with varying chemical linker length and specificity have been described previously (Fulton & Stoddart, 2001; Houseman & Mrksich, 2002; Ortiz Mellet et al., 2002). The present study introduces two new types of oligosaccharide-CD conjugates: (1) the tetrasaccharide LNnT was linked through a β-glycosylamide bond to oxidized γ-CD carboxyl-groups; and (2) nonmodified reducing LNnT was linked by oxime linkage to γ-CD which carries esterified aminooxyacetic acid units.

Aminooxy nucleophiles reacting with aldehydes or ketones result in formation of oxime bond (Rose, 1994). Synthesis of several glycopeptide analogues containing this non-natural sugar-peptide oxime-linkage has been reported previously (Marcaurelle, Rodriguez & Bertozzi, 1998; Marcaurelle, Shin, Goon & Bertozzi, 2001; Peri, Cipolla, La Ferla, Dumy & Nicotra, 1999; Peri, Dumy & Mutter, 1998; Renaudet & Dumy, 2001; Rodriguez, Marcaurelle & Bertozzi, 1998; Singh, Renaudet, Defrancq & Dumy, 2005). Most of these conjugates were prepared by conjugating aminooxy sugar analogues (sugar-α or β-ONH₂) (Cao, Tropper & Roy, 1995; Marcaurelle et al., 1998; Renaudet & Dumy, 2001; Rodriguez et al., 1998; Rodriguez, Winans, King & Bertozzi, 1997) to modified peptides presenting ketone/aldehyde groups. Alternatively, keto function present on C-glycosyl carbohydrate analogue was coupled to aminooxy-functionalized peptide backbone (Peri et al., 1999) or reducing carbohydrates were coupled to a peptide substrate containing an N,O-disubstituted hydroxylamine group (Peri et al., 1998). In addition, Boc-aminooxyacetic acid (Boc-Aoa) can be used to introduce hydroxylamine functionality to various carriers (Brask & Jensen, 2000). The present study shows that γ-CD was effectively esterified with Boc-Aoa and, after Boc removal, unprotected reducing LNnT was bound by oxime linkage in good yield to the modified γ-CD.

Both methods employed here to bind oligosaccharide groups to the γ-CD scaffold were moderately efficient, yielding products carrying on average 2-3 oligosaccharide units with the glycosylamide method and 3-4 units with the oxime linkage method. This substitution level may actually be quite sufficient in many applications. For example, it has been reported that certain cyclic peptides carrying two or three sialylated oligosaccharide ligands bind efficiently influenza hemagglutinin (Ohta et al., 2003). In addition, other studies have shown that a higher number of ligands in multivalent conjugates does not necessarily lead to increased affinity (Kalovidouris et al., 2003; Thoma, Duthaler, Magnani & Patton, 2001). These studies show that the mode of ligand presentation is crucial for generation of efficient inhibitor molecules. Therefore, it may be necessary to test several scaffold types as well as various linking methodologies for optimal multivalent product.

It has been reported that peptide-oximes, while stable under mildly acidic and neutral conditions, are unstable at high pH (Rose, 1994; Shao & Tam, 1995). If orally administered, oxime linked molecules experience highly acidic conditions in the stomach (pH˜1). At this pH, we found the half-life of approximately 3 hours for LNnT-Aoa. Even at +37° C. pH 0 half-life of about 1 hour was observed. The residence time of compounds in the stomach has been reported to be as low as 0.5 h (Sakkinen, Marvola, Kanerva, Lindevall, Ahonen & Marvola, 2006). Thus, the stability of the oxime bond in general is expected to be sufficient for therapeutic gastric applications. The oxime linked conjugate prepared in the present study however contains an ester linkage and is probably degraded by intestinal esterases.

LNnT used for conjugation in the present study is an established Helicobacter pylori binding epitope (Miller-Podraza et al., 2005). H. pylori persistently infects the gastric mucosa of a majority of the global human population. It is implicated in several diseases of the gastrointestinal tract including chronic gastritis, gastric and duodenal ulcers, and gastric adenocarcinoma (Israel & Peek, 2001; Peek & Blaser, 2002). We have here described synthesis of multivalent molecules based on linear (chondroitin 14-mer) and cyclic (γ-CD) scaffolds both presenting this established Helicobacter receptor.

### REFERENCES

Baisch, G. & Ohrlein, R. (1998). Glycosyl-transferase catalyzed assemblage of sialyl-Lewis(x)-saccharopeptides. Bioorganic and Medicinal Chemistry, 6(10), 1673-1682.
Brask, J. & Jensen, K. J. (2000). Carbopeptides: Chemoselective ligation of peptide aldehydes to an aminooxy-functionalized D-galactose template. Journal of Peptide Science, 6(6), 290-299.
Cao, S., Tropper, F. D. & Roy, R. (1995). Stereoselective phase transfer catalyzed syntheses of glycosyloxysuccinimides and their transformations into glycoprobes. Tetrahedron, 51(24), 6679-6686.
Chiu, M. H., Thomas, V. H., Stubbs, H. J. & Rice, K. G. (1995). Tissue targeting of multivalent Le(x)-terminated N-linked oligosaccharides in mice. Journal of Biological Chemistry, 270(41), 24024-24031.
Davis, B. G. (2000). Recent developments in oligosaccharide synthesis. Journal of the Chemical Society-Perkin Transaction 1(14), 2137-2160.
Dondoni, A., Marra, A., Scherrmann, M. C., Casnati, A., Sansone, F. & Ungaro, R. (1997). Synthesis and properties of O-glycosyl calix[4]arenes (calixsugars). Chemistry-A European Journal, 3, 1774-1782.
Dwek, R. A. (1996). Glycobiology: Toward understanding the function of sugars. Chemical Reviews, 96(2), 683-720.
Fraschini, C. & Vignon, M. R. (2000). Selective oxidation of primary alcohol groups of beta-cyclodextrin mediated by 2,2,6,6-tetramethylpiperidine-1-oxyl radical (TEMPO). Carbohydrate Research, 328(4), 585-589.
Fulton, D. A. & Stoddart, J. F. (2001). Neoglycoconjugates based on cyclodextrins and calixarenes. Bioconjugate Chemistry, 12(5), 655-672.
Gabius, H. J. (1997). Animal lectins. European Journal of Biochemistry, 243(3), 543-576.
Gabius, H. J., Andre, S., Kaltner, H. & Siebert, H. C. (2002). The sugar code: Functional lectinomics. Biochimica et Biophysica Acta, 1572(2-3), 165-177.
Gambaryan, A. S., Tuzikov, A. B., Chinarev, A. A., Juneja, L. R., Bovin, N. V. & Matrosovich, M. N. (2002). Polymeric inhibitor of influenza virus attachment protects mice from experimental influenza infection. Antiviral Research, 55(1), 201-205.
Houseman, B. T. & Mrksich, M. (2002). Model systems for studying polyvalent carbohydrate binding interactions. Topics in Current Chemistry, 218, 1-44.
Israel, D. A. & Peek, R. M. (2001). Pathogenesis of Helicobacter pylori-induced gastric inflammation. Alimentary Pharmacology & Therapeutics, 15(9), 1271-1290.
Kalovidouris, S. A., Blixt, O., Nelson, A., Vidal, S., Turnbull, W. B., Paulson, J. C. & Stoddart, J. F. (2003). Chemically defined sialoside scaffolds for investigation of multivalent interactions with sialic acid binding proteins. Journal of Organic Chemistry, 68(22), 8485-8493.
Lindahl, U., Li, J. P., Kusche-Gullberg, M., Salmivirta, M., Alaranta, S., Veromaa, T., Emeis, J., Roberts, I., Taylor, C., Oreste, P., Zoppetti, G., Naggi, A., Toni, G. & Casu, B. (2005). Generation of “neoheparin” from E. coli K5 capsular polysaccharide. Journal of Medicinal Chemistry, 48(2), 349-352.
Lis, H. & Sharon, N. (1998). Lectins: Carbohydrate-specific proteins that mediate cellular recognition. Chemical Reviews, 98(2), 637-674.
Manger, I. D., Rademacher, T. W. & Dwek, R. A. (1992). 1-N-glycyl beta-oligosaccharide derivatives as stable intermediates for the formation of glycoconjugate probes. Biochemistry, 31(44), 10724-10732.
Marcaurelle, L. A., Rodriguez, E. C. & Bertozzi, C. R. (1998). Synthesis of an oxime-linked neoglycopeptide with glycosylation-dependent activity similar to its native counterpart. Tetrahedron Letters, 39, 8417-8420.
Marcaurelle, L. A., Shin, Y., Goon, S. & Bertozzi, C. R. (2001). Synthesis of oxime-linked mucin mimics containing the tumor-related T(N) and sialyl T(N) antigens. Organic Letters, 3(23), 3691-3694.
Matsuda, K., Inazu, T., Haneda, K., Mizuno, M., Yamanoi, T., Hattori, K., Yamamoto, K. & Kumagai, H. (1997). The chemo-enzymatic synthesis and evaluation of oligosaccharide-branched cyclodextrins. Bioorganic & Medicinal Chemistry Letters, 7(18), 2353-2356.
Miller-Podraza, H., Lanne, B., Ångstrom, J., Teneberg, S., Milh, M. A., Jovall, P. A., Karlsson, H. & Karlsson, K. A. (2005). Novel binding epitope for Helicobacter pylori found in neolacto carbohydrate chains: structure and cross-binding properties. Journal of Biological Chemistry, 280(20), 19695-19703.
Nagasawa, K., Inoue, Y. & Tokuyasu, T. (1979). An improved method for the preparation of chondroitin by solvolytic desulfation of chondroitin sulfates. Journal of Biochemistry (Tokyo), 86(5), 1323-1329.
Ofek, I. & Doyle, R. J. (1994). Bacterial adhesion to cells and tissues. New York, Chapman & Hall.
Ohta, T., Miura, N., Fujitani, N., Nakajima, F., Niikura, K., Sadamoto, R., Guo, C.-T., Suzuki, T., Suzuki, Y., Monde, K. & Nishimura, S.-I. (2003). Glycotentacles: Synthesis of cyclic glycopeptides, toward a tailored blocker of influenza virus hemagglutinin. Angewandte Chemie International Edition, 42(42), 5186-5189.
Ortiz Mellet, C., Defaye, J., Fernandez, J. M. G. (2002). Multivalent cyclooligosaccharides: Versatile carbohydrate clusters with dual role as molecular receptors and lectin ligands. Chemistry—A European Journal, 8(9), 1982-1990.
Peek, R. M. & Blaser, M. J. (2002). Helicobacter pylori and gastrointestinal tract adenocarcinomas. Nature Reviews Cancer, 2(1), 28-37.
Peri, F., Cipolla, L., La Ferla, B., Dumy, P. & Nicotra, F. (1999). A highly convergent approach to O- and N-linked glycopeptide analogues. Glycoconjugate Journal, 16(8), 399-404.
Peri, F., Dumy, P. & Mutter, M. (1998). Chemo- and stereoselective glycosylation of hydroxylamino derivates: A versatile approach to glycoconjugates. Tetrahedron, 54, 12269-12278.
Pumphrey, C. Y., Theus, A. M., Li, S., Parrish, R. S. & Sanderson, R. D. (2002). Neoglycans, carbodiimide-modified glycosaminoglycans: A new class of anticancer agents that inhibit cancer cell proliferation and induce apoptosis. Cancer Research, 62(13), 3722-3728.
Renaudet, O. & Dumy, P. (2001). Expedient synthesis of aminooxylated-carbohydrates for chemoselective access of glycoconjugates. Tetrahedron Letters, 42, 7575-7578.
Renkonen, O., Toppila, S., Penttila, L., Salminen, H., Helin, J., Maaheimo, H., Costello, C. E., Turunen, J. P. & Renkonen, R. (1997). Synthesis of a new nanomolar saccharide inhibitor of lymphocyte adhesion: Different polylactosamine backbones present multiple sialyl Lewis x determinants to L-selectin in high-affinity mode. Glycobiology, 7(4), 453-461.
Rodriguez, E. C., Marcaurelle, L. A. & Bertozzi, C. R. (1998). Aminooxy-, hydrazide-, and thiosemicarbazide-functionalized saccharides: Versatile reagents for glycoconjugate synthesis. Journal of Organic Chemistry, 63(21), 7134-7135.
Rodriguez, E. C., Winans, K. A., King, D. S. & Bertozzi, C. R. (1997). A Strategy for the chemoselective synthesis of O-linked glycopeptides with native sugar-peptide linkages. Journal of American Chemical Society, 119, 9905-9906.
Rose, K. (1994). Facile synthesis of homogeneous artificial proteins. Journal of American Chemical Society, 116, 30-33.
Roy, R. (1996). Syntheses and some applications of chemically defined multivalent glycoconjugates. Current Opinion in Structural Biology, 6(5), 692-702.
Roy, R., Hernandez-Mateo, F. & Santoyo-Gonzalez, F. (2000). Synthesis of persialylated beta-cyclodextrins. Journal of Organic Chemistry, 65(25), 8743-8746.
Rockendorf, N. & Lindhorst, T. K. (2001). Glycodendrimers. Topics in Current Chemistry, 217, 201-238
Sakagami, M., Horie, K., Nakamoto, K., Kawaguchi, T. & Hamana, H. (2000). Synthesis of sialyl Lewis X-polysaccharide conjugates. Chemical & Pharmaceutical Bulletin (Tokyo), 48(9), 1256-1263.
Sakkinen, M., Marvola, J., Kanerva, H., Lindevall, K., Ahonen, A. & Marvola, M. (2006). Are chitosan formulations mucoadhesive in the human small intestine? An evaluation based on gamma scintigraphy. International Journal of Pharmaceuticals, 307(2), 285-291.
Satomaa, T., Toppila, S., Helin, J., Aitio, O., DeVries, A. L., Renkonen, R. & Renkonen, O. (2000). Enzymatic synthesis of a tetravalent mucin-type sialyl lewis X glycopeptide that inhibits lymphocyte adhesion to lymph node high endothelial venules. International Carbohydrate Symposium, Hamburg, Germany. Abstracts of the 20th International Carbohydrate Symposium.
Schadel, U., Sansone, F., Casnati, A. & Ungaro, R. (2005). Synthesis of upper rim calix[4]arene divalent glycoclusters via amide bond conjugation. Tetrahedron, 61, 1149-1154.
Sekiya, S., Wada, Y. & Tanaka, K. (2005). Derivatization for stabilizing sialic acids in MALDI-MS. Analytical Chemistry, 77(15), 4962-4968.
Shao, J. & Tam, J. P. (1995). Unprotected peptides as building blocks for the synthesis of peptide dendrimers with oxime, hydrazone, and thiazolidine linkages. Journal of American Chemical Society, 117, 3893-3899.
Simon, P. M., Goode, P. L., Mobasseri, A. & Zopf, D. (1997). Inhibition of Helicobacter pylori binding to gastrointestinal epithelial cells by sialic acid-containing oligosaccharides. Infection and Immunity, 65(2), 750-757.
Singh, Y., Renaudet, O., Defrancq, E. & Dumy, P. (2005). Preparation of a multi-topic glycopeptide-oligonucleotide conjugate. Organic Letters, 7(7), 1359-1362.
Soltés, L., Mendichi, R., Machovà, E., Steiner, B., Alföldi, J., Sasinkovà, V., Bystricý S. & Balog, K. (1999). Cyclodextrin derivative of hyaluronan. Carbohydrate Polymers 39(1), 17-24
Tamura, T., Wadhwa, M. S. & Rice, K. G. (1994). Reducing-end modification of N-linked oligosaccharides with tyrosine. Analytical Biochemistry, 216(2), 335-344.
Thoma, G., Duthaler, R. O., Magnani, J. L. & Patton, J. T. (2001). Nanomolar E-selectin inhibitors: 700-fold potentiation of affinity by multivalent ligand presentation. Journal of American Chemical Society, 123(41), 10113-10114.
Varki, A. (1993). Biological roles of oligosaccharides: All of the theories are correct. Glycobiology, 3(2), 97-130.
Volpi, N. (2003). Milligram-scale preparation and purification of oligosaccharides of defined length possessing the structure of chondroitin from defructosylated capsular polysaccharide K4. Glycobiology, 3(9), 635-640.
Wong, S. Y., Manger, I. D., Guile, G. R., Rademacher, T. W. & Dwek, R. A. (1993). Analysis of carbohydrate-protein interactions with synthetic N-linked neoglycoconjugate probes. Biochemical Journal, 296 (Pt 3), 817-825.

### Example 4

### Chondroitin Sulphate

### Desulphation and Acid Hydrolysis of Chondroitin Sulphate

Desulphation Nagasawai et al (1979) and hydrolysis of chondroitin sulphate A (CSA) were carried out as follows: Pyridinium salt of CSA was desulphated in dimethyl sulphoxide (DMSO) containing 10% of methanol, incubated for 5 hours at 80° C. Reaction mixture was diluted with water to DMSO concentration <5% (v/v) and pH was adjusted to 9.0-9.5 with NaOH. The mixture was dialyzed (CelluSep MWCO 6000-8000) against running tap-water for 5 hours and then against distilled water overnight. The dialyzed desulphated CS was dried by rotary evaporator. Desulphated CS was partially hydrolyzed in 0.5 M TFA for 20 h at 60° C. Hydrolyzed chondroitin was fractionated with a column of Superdex 30 (5×95 cm) eluted with 200 mM NH₄HCO₃ and the eluent was monitored at 214 nm. Fractions were analyzed by mass spectrometry. Quantitation was performed by UV-absorbance comparison to external glucuronic acid and N-acetylglucosamine standards.

### Amidation with 1,3-diaminopropane

The glucuronic acid residues in the chondroitin 14-mer (Ch14) ([GlcAβ1-3GalNAcβ1-4]₆GlcAβ1-3GalNAc) were amidated with 1,3-diaminopropane as follows: 10 μmol of chondroitin 14-mer, 7 mmol of 1,3-diaminopropane (Aldrich), 350 μmol of HBTU (Novabiochem) and 350 μmol of DIPEA (N-ethyldiisopropylamine) (Fluka Chemika) were dissolved in 40 ml of pyridine containing 10% H₂O. This mixture was stirred in the dark at RT for 3 days, and then evaporated to dryness with rotary evaporator. The reaction mixture was subjected to gel filtration chromatography in a column of Superdex 30 (5×95 cm) run in 200 mM NH₄HCO₃ and analyzed by MALDI-TOF mass spectrometry. The isolated product, amidated Ch14 (DAP-Ch14), was re-amidated with 1,3-diaminopropane due to moderate amidation level in the first reaction, and purified as described above.

### Reductive Amination

Three different oligosaccharides: LNDFH I, GnLacNAcLac, and LNnT were attached to 1,3-diaminopropane amidated chondroitin 14-mer (DAP-Ch14) by reductive amination. To 2 μmol of DAP-Ch14 35 μmol of LNDFH I or GnLacNAcLac and 0.5 mmol NaCNBH₄ were added, and each sample was dissolved in 500 μl of 0.1 M Na-borate pH 8.5. Similarly, to 1 μmol sample of DAP-Ch14 35 μmol LNnT and 1 mmol NaCNBH₄ (Aldrich) were added and sample was dissolved in 1 ml of 0.1 M Na-borate pH 8.5. All reactions were performed at room temperature for 6 days under constant magnetic stirring. Samples were purified using Superdex 30 chromatography. Fraction contents were analyzed using MALDI-TOF MS, multivalent products were pooled and finally products were subjected to MALDI-TOF MS and NMR spectroscopy.

### Example 5

### γ-CD

### Oxidation

Selective oxidation of primary alcohol groups of γ-CD with TEMPO (2,2,6,6-tetramethylpiperidine-1-oxy radical) (Aldrich) catalysis was carried out essentially as described previously (Fraschini et al (2000). Briefly, 100 μmol of γ-CD, 20 μmol of TEMPO, and 640 μmol of NaBr were dissolved in 30 ml of 0.2 M Na-carbonate buffer, pH 10. The solution was cooled on ice and 1.28 mmol of NaClO was added in several portions. The reaction was allowed to proceed for 10 min on ice and then terminated by neutralization with 4 M HCl. The oxidized γ-CD species (ox-γ-CD) were isolated by gel filtration chromatography on a column of Superdex 30 (5×95 cm) eluted with 200 mM NH₄HCO₃. The eluent was monitored at 214 nm and selected fractions were analyzed by mass spectrometry. Quantitation of products was performed by UV-absorbance comparison to external glucuronic acid standard.

### Amidation with 1,3-diaminopropane

The oxidized γ-CD was amidated with 1,3-diaminopropane as follows: 20 μmol of ox-γ-CD, 600 μmol of HBTU, 600 μmol of DIPEA and 12 mmol of 1,3-diaminopropane were dissolved in 50 ml of pyridine containing 10% H₂O. Reaction was allowed to proceed for 3 days at RT in the dark under constant stirring. The reaction mixture was then evaporated to dryness with rotary evaporator. The amidated product (DAP-ox-γ-CD) was isolated by gel filtration and analyzed by MALDI-TOF mass spectrometry.

LNnT was attached to amidated ox-γ-CD (DAP-ox-γ-CD) by reductive amination as follows: 2.8 μmol of the DAP-ox-γ-CD, 50 μmol LNnT, and 1.5 mmol NaCNBH₄ were dissolved in 2.1 ml 0.1 M Na-borate pH 8.5. Reaction was performed at room temperature for 23 hours under constant magnetic stirring and terminated by adding 100 μl 10% acetic acid (to pH 5). The mixture was purified using Superdex Peptide gel filtration and fraction contents were verified using MALDI-TOF MS. Fractions containing multivalent products were N-acetylated with acetic anhydride and purified as above. Fraction contents were verified using MALDI-TOF MS and multivalent products were pooled. Finally, the multivalent product (LNnT-DAP-ox-γ-CD) was analyzed using MALDI-TOF MS and NMR spectroscopy.

### α-2,6-sialylation

The LNnT-DAP-ox-γ-CD conjugate was sialylated using α2,6-sialyltransferase (rat; recombinant, S. frugiperda) (Calbiochem). 10 nmol of LNnT-DAP-ox-γ-CD conjugate containing on average 3 LNnT units per molecule was dissolved in 10 μl of 50 mM MES buffer (morpholinoethane sulphonate), pH 6.0, containing 640 nmol CMP-Neu5Ac (Kyowa Hakko), 5 μg bovine serum albumin (Sigma), 0.1% Triton X-100 and 0.02% NaN₃. 0.45 mU of α2,6-sialyltransferase was added and the reaction was allowed to proceed for 64 h at 37° C. The reaction was terminated by boiling for 3 minutes and purified using Superdex Peptide chromatography.

### Example 6

### Modification of LNnT Using Aminooxyacetic Acid and Amidation to DAP-ox-γ-CD

LNnT was modified using aminooxyacetic acid (Aoa) as follows: 100 μmol LNnT dissolved in 2 ml 0.2 M Na-acetate buffer, pH 4.0 and 200 μmol aminooxyacetic acid (Sigma) dissolved in 400 μl of buffer were combined and incubated at room temperature for 48 hours. The sample was purified using gel filtration chromatography in a column of Superdex 30 (5×95 cm) run in 200 mM NH₄HCO₃ and used without further purification. The DAP-ox-γ-CD was amidated with LNnT-Aoa as follows: 5 μmol of DAP-ox-γ-CD, 500 μmol DIPEA, 500 μmol HBTU, and 50 μmol LNnT-Aoa (a mixture containing 50 μmol LNnT-Aoa and 50 μmol LNnT) were dissolve in pyridine containing 10% H₂O. The reaction was performed at room temperature, in the dark, and under constant magnetic stirring for three days. The reaction mixture was evaporated to dryness with rotary evaporator and purified using Superdex 30 (5×95 cm) run in 200 mM NH₄HCO₃.

The multivalent products were isolated with gel filtration chromatography and fraction contents were analyzed using MALDI-TOF MS. Finally, pooled multivalent product (LNnT-Aoa-DAP-ox-γ-CD) was analyzed using MALDI-TOF MS in the linear negative ion mode. The indicated representative signals were identified as LNnT₁-Aoa₂-DAP₄-ox₇-γ-CD (m/z 2493 [M-3H+2Na]⁻), LNnT₂-Aoa₃-DAP₄-ox₇-γ-CD (m/z 3214 [M-H]⁻), and LNnT₃-Aoa₃-DAP₅-ox₆-γ-CD (m/z 3950 [M-H]⁻) (all proposed structures). The heterogeneity in the spectrum is due to variable levels of oxidation and amidation.

### Results and discussion for examples 4-6

### Results

### Generation of Chondroitin 14-mer

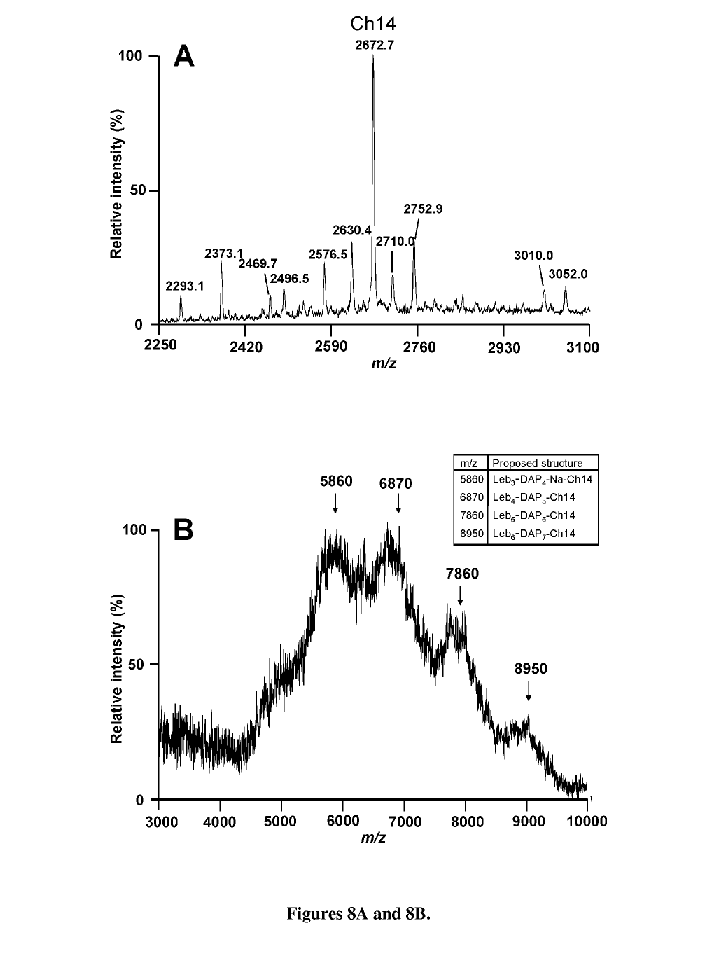

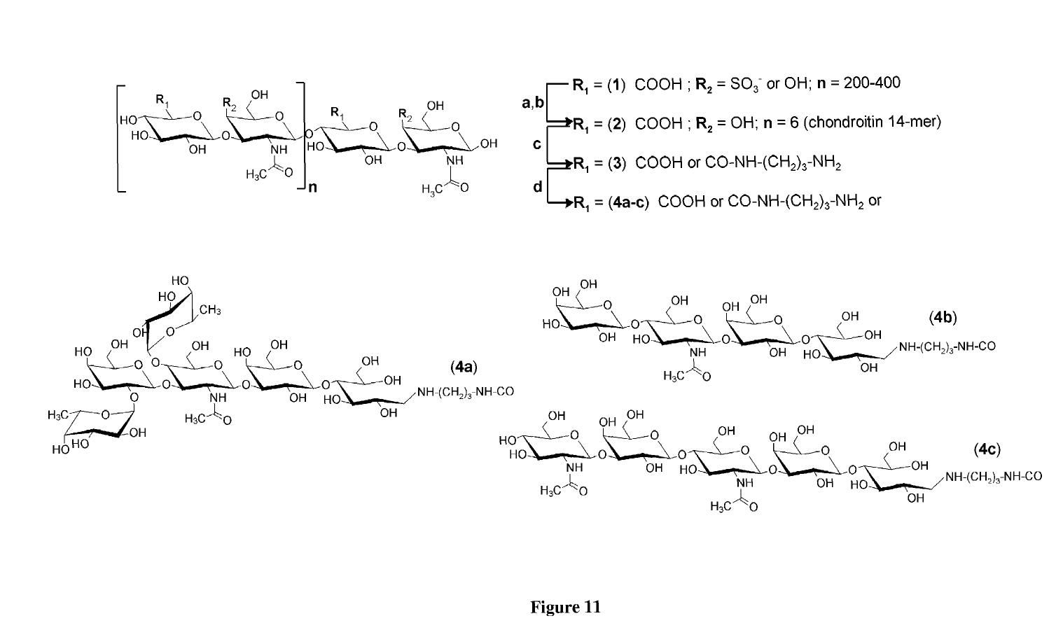

To construct a linear multivalent molecule, chemically modified chondroitin sulphate A oligomer was created to act as a carrier. First, to produce a chondroitin oligomer mixture, chondroitin sulphate A was desulphated and hydrolyzed (Scheme 3a,b) as described in Examples 4-6. The hydrolysate was fractionated by gel filtration. Mass spectrometry was used to verify fraction contents, and fractions containing 10-16-mers were pooled and re-fractionated as above. Fractions containing chondroitin 14-mer as the major compound were pooled and this fraction (Ch14, Compound 2 of FIG. 11) was again subjected to MALDI-TOF MS analysis (FIG. 8A). The MS analysis revealed that the acid hydrolysis of chondroitin resulted mainly in even-numbered oligosaccharides, i.e. oligomers composed of the repeating disaccharide unit (-4GlcAβ1-3GalNAcβ1-). All major oligomers studied were found by ¹H-NMR studies to carry a GalNAc unit at the reducing end indicating that the GalNAcβ glycosidic linkage is more susceptible to acid hydrolysis than the GlcAβ linkage. The MS analysis also showed that the desulphation was not complete but some sulphate units were still observed in the oligomers. Higher level of desulphation was not attempted as sulphation was not expected to interfere with the oligosaccharide conjugation reactions. In addition, minor de-N-acetylated species were observed but due to their low amount they were not expected to participate in subsequent reactions.

### 1,3-diaminopropane amidation of chondroitin 14-mer

Primary amine groups were introduced to chondroitin 14-mer (Ch14, Compound 2 of FIG. 11) by amidation of 1,3-diaminopropane (DAP) to glucuronic acid 6′-carboxyl groups (Scheme 3c). Reaction mixture was fractionated using gel filtration and the fractions were analyzed by mass spectrometry (data not shown). Fractions containing DAP-amidated Ch14 (Compound 3 of FIG. 11) were combined and analyzed by ¹H NMR (data not shown). The average DAP substitution level was 4.5 DAP-units per Ch14 molecule. This was calculated by comparing the integrated intensities of GalNAc βH1 and GlcA βH1 signals (4.491-4.557 ppm) to the intensity of innermost DAP methylene group signal (NH₂CH₂CH₂CH₂NH₂) (1.9 ppm). Two successive reactions were performed, each containing 100-fold molar excess of 1,3-diaminopropane and 5-fold molar excess of HBTU per glucuronic acid unit. It is possible that higher amount of these reagents could have yielded higher amidation level. Alternatively, the use of other carboxylic acid activators, like DMTMM Sekiya et al (2005) or HBPyU (Baisch & Ohrlein, 1998) instead of HBTU could be beneficial.

### Conjugation of LNDFH I, LNnT, and GnLacNAcLac to DAP-Ch14

LNDFH I, LNnT, or GnLacNAcLac were linked to DAP-Ch14 (Scheme 3d) by reductive amination. Reaction mixtures were fractionated using gel filtration, and fraction contents were verified using MALDI-TOF MS. MALDI-TOF mass spectrum of LNDFH I-DAP-Ch14 (Compound 4a of FIG. 11) showed that 2-6 oligosaccharides were attached to DAP-Ch14 backbone (FIG. 8B). Similarly, LNnT-DAP-Ch14 (Compound 4b of FIG. 11) and GnLacNAcLac-DAP-Ch14 (Compound 4c of FIG. 11) contained 2-6 oligosaccharides attached to DAP-Ch14 backbone, as analyzed by MALDI-TOF MS (data not shown).

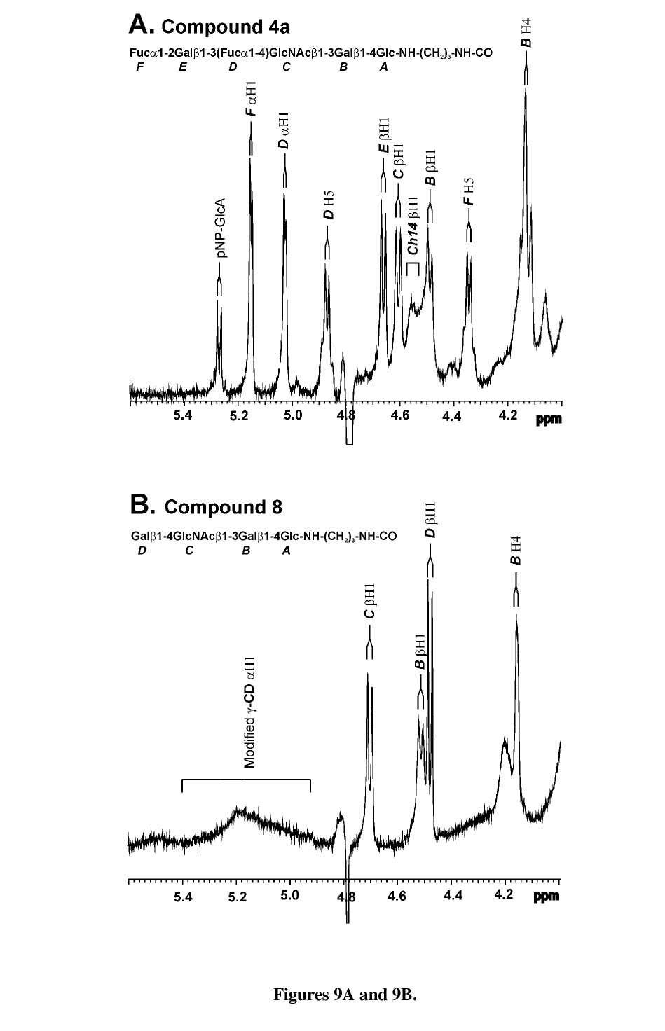

The ¹H NMR spectrum of LNDFH I linked to DAP-Ch14 backbone (LNDFH I-DAP-Ch14, Compound 4a of FIG. 11) (FIG. 9A) (carrying 100 nmol pNP-β-GlcA as internal standard, see below), show in the anomeric region H-1 resonances αH1 of F-Fuc (5.153 ppm), αH1 of D-Fuc (5.027 ppm), βH1 of E-Gal (4.662 ppm), βH1 of C-GlcNAc (4.607 ppm) consistent with those reported for the free LNDFH I molecule. In addition, H4 of 3-substituted B-Gal at 4.134 ppm, H5 of D-Fuc at 4.871 ppm, H5 of F-Fuc at 4.344 ppm, and α₃ and βCH₃ of both D- and F-Fuc were consistent with the structure. The βH1 of B-Gal had shifted downfield, from 4.416 ppm to 4.490 ppm due to reductive amination of adjacent A-Glc. All βH1 signals that originate from the chondroitin oligomer monosaccharide units can be seen resonating approximately between 4.4-4.6 ppm. Importantly, the α/βH1 of A-Glc signals are missing indicating that no reducing LNDFH I was present in the sample.

Adding an exact amount of an internal quantitation molecule (not overlapping with critical sample signals) to the NMR analysis yields a set of signals that can be integrated. These areas are easily compared to those of selected sample signals and thus reliable quantitation can be accomplished. Here, pNP-β-GlcA was added as a quantitation standard to a sample of multivalent product. pNP-β-GlcA yields signals at 5.271 ppm, 7.255 ppm, and 8.270 ppm, which do not interfere with the product signals. The average substitution level was 4.6 LNDFH I oligosaccharides per DAP-Ch14 molecule, as calculated by comparing the integrated intensities of GalNAc N-acetyl proton and LNDFH I C-GlcNAc N-acetyl proton signals. This implies that the reductive amination reaction was essentially complete as the average DAP substitution level was 4.5 (see above).

Correspondingly the ¹H NMR spectrums of LNnT-DAP-Ch14 and GnLacNAcLac-DAP-Ch14 (Compound 4b and 4c of FIG. 11, respectively) showed that βH₁ B-Gal signal had shifted downfield due to reductive amination of adjacent A-Glc and no signals was present for α/βH1 A-Glc (data not shown). This indicated that no reducing oligosaccharides remained in the sample.

### Oxidation and 1,3-diaminopropane amidation of γ-cyclodextrin

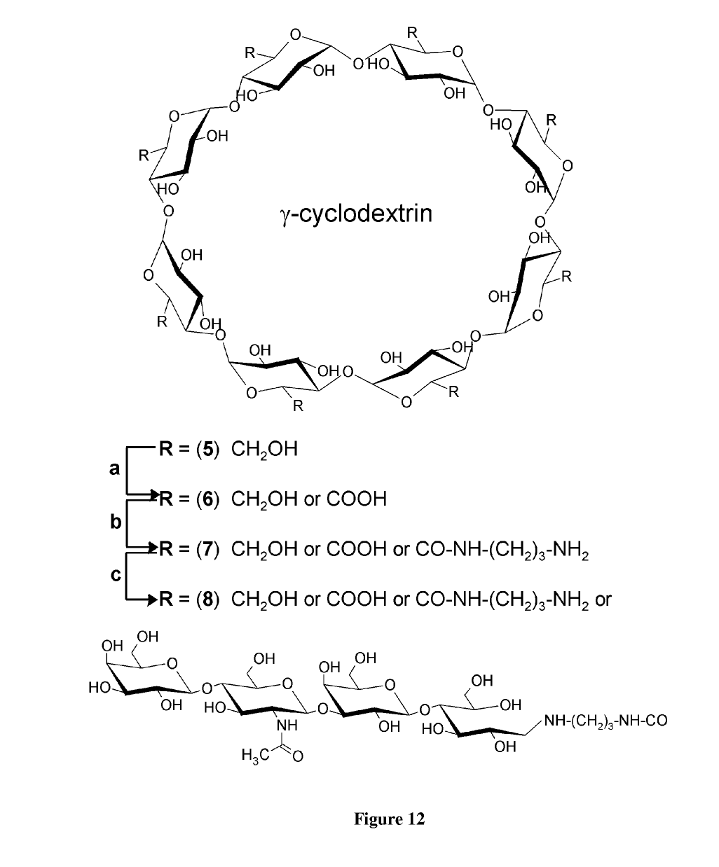

Carboxylic acid groups were introduced to γ-CD by TEMPO catalyzed oxidation (Fraschini et al (2000) (Scheme 4a of FIG. 12). A mixture of mono- to heptacarboxy-γ-CD was obtained and fractionated using gel filtration. Fraction contents were verified using MALDI-TOF MS and fractions containing penta- to heptacarboxy γ-CD were combined yielding a product (ox-γ-CD, compound 6 of FIG. 12) with an average of 6 carboxylate groups as analyzed using MALDI-TOF MS (data not shown).

Primary amine groups were introduced to oxidized γ-CD (ox-γ-CD, Compound 6 of FIG. 12) by amidation of 1,3-diaminopropane (DAP) to 6′-position carboxyl-groups (Scheme 4b) in a reaction containing DIPEA and HBTU. Reaction mixture was fractionated using gel filtration. Fraction contents were analyzed by mass spectrometry and fractions containing 1-5 DAP units were combined. The average DAP substitution level in this fraction (DAP-ox-γ-CD) was 2.5-3 (data not shown).

### Conjugation of LNnT to DAP-ox-γ-CD

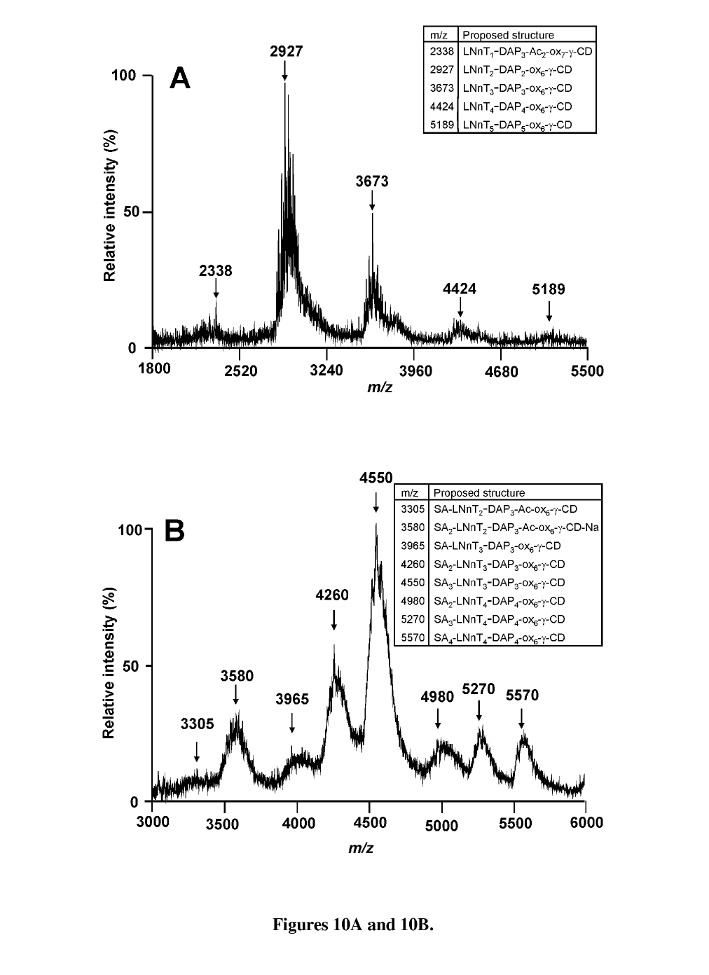

LNnT was reductively aminated to DAP amidated ox-γ-CD (DAP-ox-γ-CD, Compound 7, Scheme 4c of FIG. 12) in a buffered system. Reaction mixture was fractionated using gel filtration. Fraction contents were verified by MALDI-TOF MS. The isolated multivalent product was N-acetylated to eliminate remaining amino groups and the end product was purified again using gel filtration. Fraction contents were analyzed by MALDI-TOF MS and multivalent products were pooled. The multivalent product (LNnT-DAP-ox-γ-CD, Compound 8 of FIG. 12) was subjected to MALDI-TOF MS (FIG. 10A).

To further verify and elucidate the structure of the synthesized multivalent product, ¹H-NMR analysis was performed. The resonances of the structural reporter groups observed LNnT-DAP-ox-γ-CD (Compound 8 of FIG. 12) (FIG. 9B) show in the anomeric region αH1 resonances of the modified γ-CD around 5.166 ppm. When compared to the spectrum of unmodified γ-CD where αH1 signals (Glcα1-4) resonate at the same frequency (5.09 ppm), the αH-1 signal area of LNnT-DAP-ox-γ-CD is very heterogenous due to the complex nature of the molecule. The βH1 signals for Galβ1-4GlcNAcβ1-3Galβ1-4Glc (LNnT) C-GlcNAc at 4.703 ppm and D-Gal at 4.479 ppm at the β-anomeric region were found to be consistent with those reported for the free molecule as was H4 of 3-substituted B-Gal at 4.157 ppm. The βH1 signal for B-Gal when compared to free molecule had shifted downfield from 4.435 ppm to 4.513 ppm due to reductive amination of adjacent A-Glc. The α/βH-1 signals of A-Glc are missing indicating that no free reducing LNnT remains in the sample. In addition, the methylene signals of 1,3-diaminopropane, NAc-group signals of both C-GlcNAc and N-acetylated DAP were observed at the expected ppm-values (data not shown).

### α2,6-sialylation of LNnT-DAP-ox-γ-CD

Many bacteria (eg. Helicobacter pylori), their toxins (eg. Cholera toxin), and viruses (eg. influenza virus) attach to host cell surface carbohydrates containing sialic acid. Therefore, it was interesting to test whether the multivalent molecules could act as an acceptor to sialyltransferase to yield sialylated multivalent conjugates. This was tested by performing α2,6-sialyltransferase reaction with (LNnT)₂₋₄-DAP-ox-γ-CD, which contains terminal β1-4 linked galactose residues serving as possible acceptors. (LNnT)₂₋₄-DAP-ox-γ-CD was incubated with CMP-Neu5Ac and α2,6-sialyltransferase as described in above. The sialylated product (SA-LNnT-DAP-ox-γ-CD) was isolated by gel filtration and analyzed using MALDI-TOF mass spectrometry (FIG. 10B). The major product was found to be the fully sialylated SA₃-LNnT₃-DAP-ox-γ-CD.

### Discussion

The development of carbohydrate-based anti-adhesives presents a promising approach for the prevention of microbial infections, even more so given the increasing incidence of bacterial resistance to traditional antibiotics. Natural carbohydrate ligands are in many cases presented as clusters (Crottet et al (1996), which increases the functional affinity (avidity) of monomeric carbohydrate ligands usually expressing very low affinities to their protein receptors. Therefore, artificial carbohydrate pharmaceuticals should be constructed as multivalent carbohydrates or glycoclusters Schengrund (2003), Turnbull and Stoddart (2002).

In the present study, we have conjugated by reductive amination unmodified reducing oligosaccharides (tetra-, penta- and hexasaccharides) to scaffold molecules containing free amino groups. Reductive amination is an established method in neoglycoconjugate synthesis and the reactions can be performed in the absence of protective groups on the sugar units and under aqueous conditions. Two different scaffold molecules were used in the present study: (1) a chondroitin 14-mer fraction modified to express primary amino groups and (2) γ-cyclodextrin modified to express primary amino groups. The chondroitin 14-mer fraction used in these experiments was isolated from a desulphated chondroitin sulphate acid hydrolysate by gel filtration chromatography, and primary amine groups were added by amidation of 1,3-diaminopropane to carboxyl groups. To prepare the γ-cyclodextrin scaffold, glucuronic acid units were first introduced by oxidizing the primary hydroxyl groups by TEMPO oxidation to carboxyl groups, followed by diaminopropane amidation. The amine modified scaffolds described here are versatile and effective as these can be modified by sugar ligands to create multivalent conjugates of different specificities.

GAGs are excellent scaffold candidates for constructing multivalent glycoconjugates because their carboxyl-groups can be functionalized for subsequent attachment of carbohydrate units. However, only a few studies showing GAG based oligosaccharide conjugates have been published. These include sialyl-Lewis x-heparin conjugates Sakagami et al (2000) and conversion of hyaluronan to its β-cyclodextrin derivate SOltes et al (1999). Here we prepared a chondroitin 14-mer fraction, which was used as a scaffold to which tetra-, penta-, or hexasaccharides were attached. The chondroitin oligomer based conjugates present their oligosaccharide ligands on a linear scaffold, which may mimic e.g. natural mucins and polylactosaminoglycans. Polyvalent sialyl-Lewis x conjugates based on mucin type or polylactosamine scaffolds have been shown to bind selectins with high affinity Satomaa et al (2000), Renkonen et al (1997).

Substituted cyclodextrins may present their ligands in a relatively rigid fashion, and these can be useful binders to bacterial toxins and influenza virus hemagglutinin type proteins [Kitov et al (2000), 36]. The multivalency effect of CD-carbohydrate conjugates has been previously demonstrated in several studies, e.g. Furuike et al (2000)—Andre et al (2004). Here, we constructed a novel LNnT conjugate based on γ-CD scaffold. A similar linker build by coupling ox-β-CD and carbohydrate glycosides with primary amino groups has been described previously Ichikawa et al (2000)]. The method of the present study however has the advantage of using unmodified reducing sugars, and thus it is not necessary to synthesize a glycoside of each oligosaccharide ligand.

A human milk tetrasaccharide γ-CD conjugate (LNnT-DAP-ox-γ-CD) synthesized in the present study was also effectively sialylated by a α2,6-sialyltransferase. The fact that all LNnT units could be sialylated shows that they are well available for biological recognition. Based on the structural data from influenza hemagglutinin, a chemoenzymatic approach has previously been used to construct cyclic peptide scaffolds presenting three sialotrisaccharide units and these conjugates were shown to exhibit scaffold-dependant binding affinities against hemagglutinin Ohta et al (2003)]. On the same concept, it would be of great interest to study sialyloligosaccharide conjugates based on cyclic carbohydrate based scaffolds (α-, β-, or γ-CD).

All oligosaccharides used for conjugation in the present study are established Helicobacter pylori binding epitopes [40, Miller-Podraza et al (2005). It has previously been shown that high doses of antiadhesive carbohydrates could cure Helicobacter pylori in Rhesus monkeys Mysore et al (1999). However, monovalent carbohydrate molecules are generally weak binders, and therefore it will be of great interest to assess the H. pylori binding activity of the present conjugates.

### Example 7

Hyaluronic acid is amidated with 1,3-diaminopropane as follows: 10 μmol of hyaluronic acid, 7 mmol of 1,3-diaminopropane (DAP), 350 μmol of HBTU and 350 μmol of DIPEA (N-ethyldiisopropylamine) are dissolved in 40 ml of pyridine containing 10% H₂O. This mixture is stirred in the dark at RT for 3 days, and is then evaporated to dryness with a rotary evaporator. The amidated hyaluronic acid is purified by gel filtration chromatography in a column of Superdex 30 (5×95 cm) run in 200 mM NH₄HCO₃ and analyzed by NMR spectroscopy.

The DAP amidated hyaluronic acid is derivatized with a reducing carbohydrate by reductive amination as follows: The DAP amidated hyaluronic acid and the reducing carbohydrate are dissolved in 0.1 M Na-borate pH 8.5 containing 0.5 mmol NaCNBH₄ and the reaction is allowed to proceed for 1-6 days. The modified hyaluronic acid product is isolated by Superdex 30 chromatography.

### Example 8

Dermatan sulphate oligomer is amidated with an oligosaccharide glycosylamine as follows: Dermatan sulphate oligomer (150 nmol), LNnT-NH₂ (10 μmol), HBTU (10 μmol) and DIPEA (N-ethyldiisopropylamine) (10 μmol) are dissolved in dry pyridine (2.35 ml). Reaction is allowed to proceed at room temperature in the dark for four days. Reaction mixture is then dried in a rotary evaporator, followed by addition of 5 ml of methanol and evaporation repeated three times. The modified dermatan sulphate oligomer product is isolated by Superdex 30 chromatography.

### Abbreviations Used

Ch14, chondroitin 14-mer; CMP-Neu5Ac, cytidine 5′-monophospho-5-N-acetyl neuraminic acid); CS, chondroitin sulphate; DAP, 1,3-diaminopropane; DAP-Ch14, 1,3-diaminopropane amidated chondroitin 14-mer; DAP-ox-γ-CD, oxidized and 1,3-diaminopropane amidated γ-cyclodextrin; DIPEA, N-ethyldiisopropylamine; DMSO, dimethyl sulphoxide; γ-CD, γ-cyclodextrin; pNP-β-GlcA, para-nitrophenyl-β-glucuronide; GnLacNAcLac, GlcNAcβ1-3Galβ1-4GlcNAcβ1-3Galβ1-4Glc; HBTU, 2-(1H-bentsotriazole-1-yl)-1,1,3,3-tetramethyluronium hexafluorophosphatel; LNDFH I, Fucα1-2Galβ1-3(Fucα1-4)GlcNAcβ1-3Galβ1-4Glc; LNnT, Galβ1-4GlcNAcβ1-3Galβ1-4Glc; MALDI-TOF MS, matrix-assisted laser desorption-ionization time-of-flight mass spectrometry; MES, morpholinoethane sulphonate; ox-γ-CD, oxidized γ-cyclodextrin; SA, sialyl; TEMPO, tetramethylpiperidine-1-oxy radical.

1. A polyvalent conjugate comprising
(i) a backbone structure (PO) of 5 to 20 monosaccharide units or of a polysaccharide,
(ii) a carbohydrate comprising, preferably oligosaccharide, biorecognition groups (Bio) of 1 to 10 monomer units,
(iii) a bifunctional spacer groups of the formula -(y)ₚ-(S)_{q}-(z)ᵣ-, wherein S is a spacer group, p, q and r are each 0 or 1, whereby at least one of p and r is different from 0, and y and z are chemoselective ligation groups, which covalently link a said Bio group to said backbone structure, and wherein the degree of conjugation being from 0.2 to 1, or a precursor comprising structure of iii) and either i) or ii).

2. The conjugate according to claim 1 wherein the conjugate is according to Formula I:
[Bio-(y)ₚ-(S)_{q}-(z)ᵣ-]ₙ[Z]ₘPO (I)
wherein PO, Bio, y, S, z, p, q and r have the meaning given above, n indicates the number of biorecognition groups in the conjugate, Z can have the meaning of (y′)ₚ-(S)_{q}-(z)ᵣ- or of a group z′, wherein y′ means a group that can form the linkage y, and z′ means a group on PO that can form the linkage z, and m is an integer which is >0 so that (n+m) is equal to or less. Preferably m is 0, meaning that the conjugate product does not essentially contain any species with incompletely reacted spacer groups or fractions thereof.

3. The conjugate according to claim 1, wherein conjugate is according the formula Ia:
{Hex2(X)ₚ₁}[aHex(X)ₚ₂b{Hex2(X)ₚ₃}ₚ₄]ₙ₁aHex(X)ₚ₅—R (Ia)
wherein Hex and Hex2 are each a hexose group which comprises a group for bonding to X, X is a bioactive conjugate according to the formula:
Bio-(y)ₚ-(S)_{q}-(z)ᵣ-
wherein Bio, S, y, z, p, q, and r have the meaning given in the claim 1, n1 is an integer >1, each of p1, p2, p3, p4 and p5 are 0 or 1, provided that at least one of p1, p2, p3, p4 and p5 is different from 0,
a and b are the anomeric linkages of the monosaccharide Hex2 and Hex respectively, the linkage positions being either α or β1-4/1-3, and R is a derivatization group at the reducing end of the saccharide, or a modified reducing end, such as reduced monosaccharide residue, an alditol, or anhydromannitol.

4. (canceled)

5. The conjugate according to claim 1, wherein conjugate is according to the formula II:
[(y′)ₚ-(S)_{q}-(z)ᵣ]ₙ-PO (II)
wherein n is an integer >1,
S is a spacer group,
y′ is an aminooxy group NH₂—O— or a chemoselective linking group, and
z is a O-hydroxylamine residue —O—NH— or O—N═,
with the nitrogen atom being linked to the PO structure, or a chemoselective linking group, there being at least one of aminooxy and O-hydroxylamine group present;
p, q and r are each 0 or 1, whereby at least one of p and r is different from 0, and PO is a linear polysaccharide or oligosaccharide or mixture thereof carrying n [(y′)ₚ-(S)_{q}-(z)ᵣ]ₙ- groups on the polymer backbone.

6. (canceled)

7. The conjugate according to the claim 5 wherein the PO backbone is
a) glycasaminoglycan selected from the group: hyaluronic acid, chodroitin (non-sulfated), chondroitin sulfates, heparan sulfates, preferably conjugated from carboxylic acid or secondary amine group
or
b) cyclodextrin conjugated from 6-position carbonyl or ester.
c) chitosan and the BIO-carbohydrate is linked from position which is not redicing end, preferably from 6 position or secondary amine.

8. The conjugate according to claim 1, wherein the conjugate is according to the formula IIa:
[NHR″O—(S)_{q}—CO—O/NH]ₙ—PO (IIa)
wherein the symbols PO, S, q and n have the meaning given above in formula (II),
q2 is an integer from 1 to 26, and
R″ is hydrogen or a N-protecting group such as N-Boc, and
O/NH indicates that the branch is linked to the polymer backbone by an amide linkage formed with an amine group on the or an ester linkage formed with a hydroxy group on the polymer backbone.

9. (canceled)

10. The conjugate according to claim 1, wherein the conjugate is according to the formula IId:
[NHR″O—(S)_{q}—CO—O/NH]ₙ-cyclodextrin (IId)
wherein the symbols R″, S, q, q2 and n have the meaning given in the formula (IIa).

11. (canceled)

12. The conjugate according to claim 1, wherein the conjugate is according to the formula III:
Carb-NR′—O—(S)_{q}-(z′)ᵣ (III)
wherein Carb is carbohydrate, such as an oligo- or monosaccharide, R′ is hydrogen or a further bond to Carb, S, q and r have the meanings given in the formula II and z′ is a group that can react with the polymer, such as the polysaccharide and/or carbohydrate backbone structure PO to form the group z, where z is as defined, for conjugation with a polymer, such as a polysaccharide and/or carbohydrate backbone structure, preferably with an carbohydrate backbone structure, and more preferably a chitosan oligomer backbone structure.

13. The conjugate according to claim 1, wherein the conjugate is according to the formula IIIa:
Carb-NR′—O—(S)_{q}—COR′″ (IIIa)
wherein the symbols S, q, q2 and n have the meaning given in the formula (IIa), and R′ is the same as in the formula (III), R′″ is OH or a carboxylic acid activating conjugate, preferably a succinimide ester.

14. (canceled)

15. (canceled)

16. (canceled)

17. The conjugate according to claim 13, wherein cyclodextrin is γ-cyclodextrin.

18. (canceled)

19. The conjugate according to claim 13, wherein glycosaminoglycan, preferably chondroitin fragment is chondroitin 10, 12, or 14 mer.

20. The conjugate according to claim 1, wherein the Bio group has the oligosaccharide sequences according to the formula:
[Hex1(A)_{q1}(NAc)ᵣ₁y3]ₛGal(NAc)ᵣ₂β4Glc(A)_{q2}(NAc)ᵣ₃
wherein q1, q2, r1, r2, r3, r5 and s, are each independently 0 or 1, and Hex1 is a hexose structure, preferably galactose (Gal) or glucose (Glc) or mannose (Man), most preferably Gal or Glc, which may be further modified by the A and/or NAc groups; y is either alpha or beta indicating the anomeric structure of the terminal monosaccharide residue, and analogs or derivatives of said oligosaccharide sequence.

21. (canceled)

22. (canceled)

23. (canceled)

24. The conjugate according to claim 20 wherein the biorecognition group is selected from the group consisting of: sialyl-Lewis X, sialyl-Lewis A, VIM-2, Lewis A, B, X, Y and Z type.sup.1, A type.sup.2, B type.sup.1, B type.sup.2 and H type.sup.1, H type.sup.2.

25. (canceled)

26. The conjugate according to claim 1, wherein the conjugate is selected from the group: a conjugate according to the formula:
[Carb-NH—CO]ₙ—PO (2b)
wherein PO is a poly- or oligosaccharide, Carb is the reducing carbohydrate converted to a glycosylamine and coupled by an amide linkage to a C═O unit originating from a carboxylic acid group of PO, and n has the meaning given in the formula (I)
or
the conjugate is according to the formula:
[Carb-CO—NH]ₙ—PO (2c)
wherein PO is a poly- or oligosaccharide, Carb is a carbohydrate carrying a carboxylic acid group and coupled by a amide linkage to a NH unit originating from an amine group of PO, and n has the meaning given in the formula (I).

27. (canceled)

28. The conjugate according to claim 1, wherein the chemoselective ligation group y and/or z is selected from the group consisting of —N—NH—, —N—NR₁—, —C(═O)—O—, —C(═O)—, —C(═O)—NH—, —O—NH—, —O—N═, —O—, —S—, —NH—, and —NR₁—, wherein R₁ is H or a lower alkyl group, preferably containing up to 6 carbon atoms.

29. (canceled)

30. (canceled)

31. (canceled)

32. A process for the preparation of the conjugates as defined in claim 1, comprising
a) reacting an oligosaccharide PO carrying a spacer group (y′)ₚ-(S)_{q}-(z)ᵣ- with a compound of the formula Bio-Y″ wherein PO, Bio, S, p, q, z, and r have the meanings given in Formula I above, Y″ is a reactive group, such as an amino, hydroxy, carboxylic acid, activated ester, aldehyde or a keto group, and y′ is a group capable of reacting with the group Y″ on the Bio group to form the linkage y, wherein y means the same as in formula I, or
b) reacting a compound having the formula Bio-(y)ₚ-(S)_{q}-(z′)ᵣ with an oligosaccharide PO having a reactive group X″, such as an amino, hydroxy, carboxylic acid, activated ester, aldehyde or a keto group, wherein PO, Bio, S, p, q, y, r and n have the meanings given in Formula I above, and z′ is a group capable of reacting with X″ to form the linkage z, wherein z means the same as above.

33. The process according to claim 32, wherein PO and Bio are oligosaccharides which are used in unprotected form, and the step of linking the oligosaccharide is performed in an aqueous solution.

34. (canceled)

35. (canceled)

36. (canceled)

37. (canceled)

38. (canceled)

39. (canceled)

40. Method for inhibiting Helicobacter pylori, or diarrhea causing Escherichia coli, or for the inhibition of binding of pathogenic bacteria, viruses or toxins to cell surface receptors in an individual in need of such inhibition, comprising administering to such an individual an effective amount of a conjugate according to the claim 1.

41. (canceled)

42. (canceled)

43. (canceled)

44. (canceled)

45. (canceled)

46. The conjugate according to the claim 1 selected from the group: functional food comprising the conjugate according or a medicament comprising the conjugate.

47. (canceled)

48. The biorecognition conjugate according to the claim 1, wherein the conjute is derived from
a first carbohydrate, preferably comprising at least one monosaccharide, or at least one oligosaccharide (including disaccharides and larger oligosaccharides from trimer to decamers) or at least one polysaccharide residue, comprising
i) at least one monosaccharide residue comprising a carbonyl group, preferably a reducing end carbonyl group or a carbonyl group linked to a furanose (or five membered) or pyranose (or six membered ring, more preferably a carbonyl group linked to a furanose or pyranose ring, and in a preferred embodiment preferably a pyranose structure of the carbohydrate
and/or
ii) at least one monosaccharide residue comprising an amine group linked to a furanose or pyranose ring. The amine is a primary amine, glycosylamine or secondary amine, preferably a primary amine, preferably on a 2-position of furanose or pyranose ring.
and
a second carbohydrate, preferably comprising at least one monosaccharide, or at least one oligosaccharide (including disaccharides and larger oligosaccharides from trimer to decamers) or at least one polysaccharide residue, comprising
a) at least one monosaccharide residue comprising a carbonyl group, preferably a reducing end carbonyl group or a carbonyl group linked to a furanose or pyranose ring, more preferably a carbonyl group linked to a furanose or pyranose ring, and in a preferred embodiment preferably a pyranose structure of the carbohydrate and/or
b) at least one monosaccharide residue comprising an amine group linked to a furanose or pyranose ring. The amine is a primary amine, glycosylamine or secondary amine, preferably a primary amine, preferably on a 2-position of furanose or pyranose ring.
and first and second carbohydrate are covalently linked (directly or through a spacer) to each other,
with the provision that at least one carbonyl group or amine group of one of the carbohydrates is linked to the carbonyl or amine group or a hydroxyl group of the other carbohydrate,
and a carbonyl group being
1) an aldehyde or ketone is changed in the conjugation to a derivative of aldehyde or ketone including oximes, Schiff bases:
and/or
2) a carboxylic acid is changed to an amide or an ester
and/or an amine is derived to
3) a Schiff base; or an amine by reduction, or amide.

49. (canceled)
